(19) United States
(12) Patent Application Publication
(10) Pub. No.: US 2011/0301218 A1
(43) Pub. Date: Dec. 8, 2011

(54) MODIFIED snRNAs FOR USE IN THERAPY

(76) Inventors: Irene Bozzoni, Rome (IT); Femanda De Angelis, Rome (IT); Tania Incitti, Rome (IT)

(21) Appl. No.: 13/025,428

(22) Filed: Feb. 11, 2011

(30) Foreign Application Priority Data
Jun. 8, 2010 (IT) RM2010A000310

(51) Int. Cl. A61K 31/7088; C07H 21/04; C07H 21/02; A61P 21/00; C12N 5/10; C12N 15/64; A61K 48/00; C07H 21/00; C12N 15/63

(52) U.S. Cl. 514/44 A; 536/23.1; 435/320.1; 435/325; 435/348; 435/69.1; 514/44 R

(57) ABSTRACT

The invention relates to a nucleic acid encoding a small nuclear RNA (snRNA), which snRNA is modified so that it contains sequence capable of hybridizing with the 5′ and 3′ splice site junctions and an exonic splicing enhancer of an exon of a pre-mRNA, so that the exon sequence is skipped during the splicing process that converts the pre-mRNA in the mature mRNA. The nucleic acid, the modified snRNA and vectors incorporating the nucleic acid may be used in the therapy, in particular in the treatment of muscular dystrophies.

The present invention claims priority to Italian Patent Application No. RM2010A000310, filed on Jun. 8, 2010, which is incorporated by reference herein in its entirety.

### FIELD OF THE INVENTION

The invention relates to a nucleic acid encoding a modified snRNA and to the snRNA encoded by the nucleic acid. The invention also relates to a vector incorporating the nucleic acid and to cells comprising the nucleic acid, the modified snRNA and the vector. The invention further relates to a method for the preparation of a parvoviral gene delivery vector and to the use of the nucleic acid, the modified snRNA and the vector in methods of therapy.

### BACKGROUND TO THE INVENTION

Duchenne Muscular dystrophy (DMD) is one of the most severe neuromuscular diseases, affecting 1:3500 live males. DMD is a monogenic disorder caused by mutations in the largest gene of higher eukaryotes, the gene encoding for the dystrophin protein. About 98% of the 2.4 Mb DMD gene accounts for intron sequences while the remainder constitutes of 79 exons and seven different promoters, which direct the expression of tissue specific isoforms. Additional isoforms also arise from alternative splicing or polyadenylation.

In skeletal muscle tissues, dystrophin is localized on the inner face of sarcolemma, the muscle fiber plasma membrane, where it interacts with the cytoskeletal actin by its N-terminal domain, and with a complex of proteins localized on the sarcolemma, named dystrophin associated protein complex (DAPC), through its C-terminal. These interactions allow muscle force transduction and are essential for fibre integrity. Several other crucial aspects of the muscle fibre physiology, like calcium homeostasis, and epigenetic control of gene expression, have recently emerged as dystrophin-dependent, providing an explanation for the dramatic consequence observed on the muscle tissue of Duchenne patients where dystrophin is absent.

Due to the well defined genetic alteration, many studies have concentrated on finding possible therapeutic approaches for the treatment of DMD. With a few exceptions, all DMD mutations (deletions and duplications) lead to frameshifts, resulting in the formation of a premature termination codon that impairs dystrophin translation. On the contrary, when mutations do not perturb the correct reading frame, as in the case of Becker Muscular Dystrophy (BMD), the translation of a partially functional protein is still occurring. Notably, BMD patients carrying deletions of about half of the DMD gene show very mild symptoms. These observations suggested a new therapeutic strategy based on the possibility of manipulating the splicing of the dystrophin precursor mRNA in order to induce the skipping of specific exons and rescuing a correct reading frame. This can be obtained by antisense molecules that, by pairing with splice junctions or exonic splicing enhancers (ESEs), prevent exon recognition by the splicing machinery.

Different groups developed chemically modified Antisense Oligonucleotides (AONs) directed against splice junctions or ESEs, and found that this strategy could effectively rescue dystrophin both in vitro and in vivo. Safety and local efficacy of the intramuscular AONs injections were tested in phase I clinical trials and systemic trials recently started.

Small Nuclear RNAs (snRNA) have also been utilized as vectors for stable antisense expression in order to circumvent the major limitation of the AON approach that, given the limited oligo stability, requires reiterative administrations. U1 and U7 snRNA-derived antisense molecules have been shown to be effective in inducing exon skipping and dystrophin rescue both in human DMD myoblasts and in the mdx mouse. Furthermore, long term and body-wide effectiveness of the exon skipping therapy was obtained by systemic injection of adeno-associated viral vector (AAV) expressing the U1-derived antisense molecules into mdx mice.

There is a need for improved molecules of this type.

### BRIEF DESCRIPTION OF THE INVENTION

A modified snRNA has been developed with the ability to target both the 5′ and 3′ splicing junctions and an exonic splicing enhancer of an exon of a gene (for example an exon of the dystrophin gene). This modified snRNA mediates highly efficient skipping of the exon. The modified snRNA may thus be used to skip exons, resulting in either removing exons carrying mutations and/or restoring the reading frame. In the absence of skipping a non-functional protein would be formed or, more generally, be totally absent. The modified snRNA may be used to skip such exons such that the resulting protein thus formed is functional.

According to the invention, there is thus provided a nucleic acid encoding a small nuclear RNA (snRNA), which snRNA is modified so that it comprises sequence capable of hybridizing with the 5′ and 3′ splice site junctions and an exonic splicing enhancer sequence of an exon of a pre-mRNA, so that the exon sequence is skipped during the splicing process that converts the pre-mRNA into a mature mRNA. The pre-mRNA may be that of dystrophin. The exon targeted may be exon 51 of the dystrophin gene.

Typically, a nucleic acid of the invention is a single molecule which comprises antisense sequence capable of targeting 5′ and 3′ splice site junctions and an exonic splicing enhancer sequence. However, it is possible to provide two or three nucleic acids so that each nucleic acid provides one or two of the antisense sequences. Such nucleic acids may then be provided at the same time to a cell so that skipping of an exon may occur.

The invention also provides a modified snRNA encoded by a nucleic acid of the invention and a vector which incorporates a nucleic acid of the invention. The invention further provides a mammalian or insect cell comprising a nucleic acid, a modified snRNA or a vector of the invention.

In addition, the invention provides a method for the preparation of a parvoviral gene delivery vector which method comprising the steps of:
(a) providing an insect cell comprising one or more nucleic acid constructs comprising:
   (i) a nucleic acid of the invention that is flanked by at least one parvoviral inverted terminal repeat nucleotide sequence;
   (ii) a first expression cassette comprising a nucleotide sequence encoding one or more parvoviral Rep proteins which is operably linked to a first promoter that is capable of driving expression of the Rep protein(s) in the insect cell;
   (iii) a second expression cassette comprising a nucleotide sequence encoding one or more parvoviral capsid proteins which is operably linked to a second promoter that is capable of driving expression of the capsid protein(s) in the insect cell;
(b) culturing the insect cell defined in (a) under conditions conducive to the expression of the Rep and the capsid proteins; and, optionally,
(c) recovery of the parvoviral gene delivery vector.

The invention also provides:

a pharmaceutical composition comprising a nucleic acid, a modified snRNA or a vector of the invention and a pharmaceutically acceptable carrier or diluent;

a nucleic acid, a modified snRNA or a vector of the invention for use in the treatment of the human or animal body by therapy; and

a method of treatment of a muscular dystrophy, which method comprises the step of administering an effective amount of a nucleic acid, a modified snRNA or a vector of the invention to a subject in need thereof.

### BRIEF DESCRIPTION OF THE DRAWINGS

FIG. 1 shows the design and cloning of the U1-antisense RNA molecules. (a) Schematic representation of the chimeric U1-antisense snRNA. The grey box indicates the location of the antisense sequences. (b) Table summarizing the 7 different constructs produced together with the corresponding target regions on exon 51 and flanking intron sequences (uppercase—exonic regions; lowercase—intron sequences).

FIG. 2 shows the U1-antisense expression in HeLa cells. (a) Schematic representation of pRRL/U1-antisense constructs. The U1-antisense expression cassettes were cloned in the dU3 region of the downstream LTR of the pRRLSIN.cPPT.PGK/GFP.WPRE lentiviral backbone. hPGK: human PhosphoGlycerate Kinase promoter; GFP: Green Fluorescent Protein cDNA. (b) HeLa cells were transfected with the different antisense constructs together with the U16-RBE plasmid (expressing a 143 nucleotide long modified U16 snoRNA). Northern blot analysis was performed with probes against: U1 snRNA (panel U1), U16-RBE (panel RBE) and U2snRNA (panel U2). The two latter hybridizations are used to normalize for transfection efficiency and as loading control, respectively. The filter was cut such as to exclude the endogenous U1 snRNA hybridization. On the side, the molecular sizes are reported. (c) Nuclear extracts were prepared from the 5′3′ esx sample from the experiment in (a) and immunoprecipitated with anti U1-70K antibodies. The RNA, extracted from the Ippt pellet, was analyzed for the presence of the 5′3′ esx transcript by RT-PCR with primers shown on the right panel.

FIG. 3 shows Exon 51 skipping and dystrophin rescue in DMD Δ48-50 primary myoblasts and selection of the best performing construct with the Luciferase-based splicing reporter assay. (a) Top: Schematic representations of the Δ48-50 Dystrophin cDNA between exons 46 and 54 together with the positions of RT-PCR oligos (E46F and E54R-RT), and Nested PCR oligos (E47F and E52Ro-nRT). Bottom: nested RT-PCR on RNA extracted from Δ48-50 cells infected with the antisense-expressing lentiviruses. Unskipped and skipped products are indicated on the right (mi: mock infected; −: negative control without RNA). Molecular sizes are indicated on the left. (b) Representative western blot of proteins (50 μg) extracted from infected Δ48-50 cells, probed with anti-dystrophin (Dys) and anti-tubulin (Tub) antibodies (WT: 2 μg of proteins from wild type skeletal muscle cells; mi: 50 μg of proteins from mock infected cells). The histogram at the bottom indicates dystrophin levels normalized on tubulin signals. Values are obtained from three independent experiments; error bars: means±SD. (c) Top: schematic representation of the pLuc-ex51 construct. Exon 51 and part of its flanking introns were cloned between the Firefly luciferase exons. From this construct, luciferase expression is obtained only upon exon 51 skipping. Bottom: Histogram showing the relative luminiscence units (RLU) from C₂7 myoblast cells transfected with pLuc-ex51 and pTK (Renilla luciferase expressing plasmid), together with either 5′3′ or 5′3′ esx constructs. An empty lentiviral vector is used as control (PGK).

FIG. 4 shows trans-differentiation of DMD Δ445-50 fibroblasts to myoblasts and dystrophin rescue upon exon 51 skipping. (a) Schematic representation of MyoD and M-U1#51 constructs. hPGK: human PhosphoGlycerate Kinase promoter; MyoD: MyoD cDNA; IRES: Internal Rybosome Entry Site; GFP: Green Fluorescent Protein cDNA. The U1 cassette expressing the 5′3′ esx construct was cloned into the dU3 region. (b) Western blot analysis with anti-dystrophin (Dys) and anti-tubulin antibodies (Tub) on proteins from MyoD-infected wild type fibroblasts (Fb WT) collected at days 0, 2, 4, 6 and 8 after induction of differentiation (C: proteins from human skeletal muscle cells). Δ45-50 fibroblasts were infected with MyoD or M-U1#51 and collected at 9 and 13 days of differentiation. Samples were analyzed by: Western blot, for MyoD and GFP expression (c), Northern blot, for antisense RNA expression (d), nested RT-PCR for exon skipping (e) and western blot for dystrophin rescue (f). Western blot with anti-tubulin (Tub) antibodies was used as a loading control.

FIG. 5 shows: (a) sequence of exon 51 and its intronic flanking regions—sequences targeted by the U1 derived 5′3′ esx antisense construct are indicated as the shaded region; (b) modified U1 snRNA 5′3′ esx sequence—the antisense sequence is indicated as the shaded region.

### BRIEF DESCRIPTION OF THE SEQUENCE LISTING

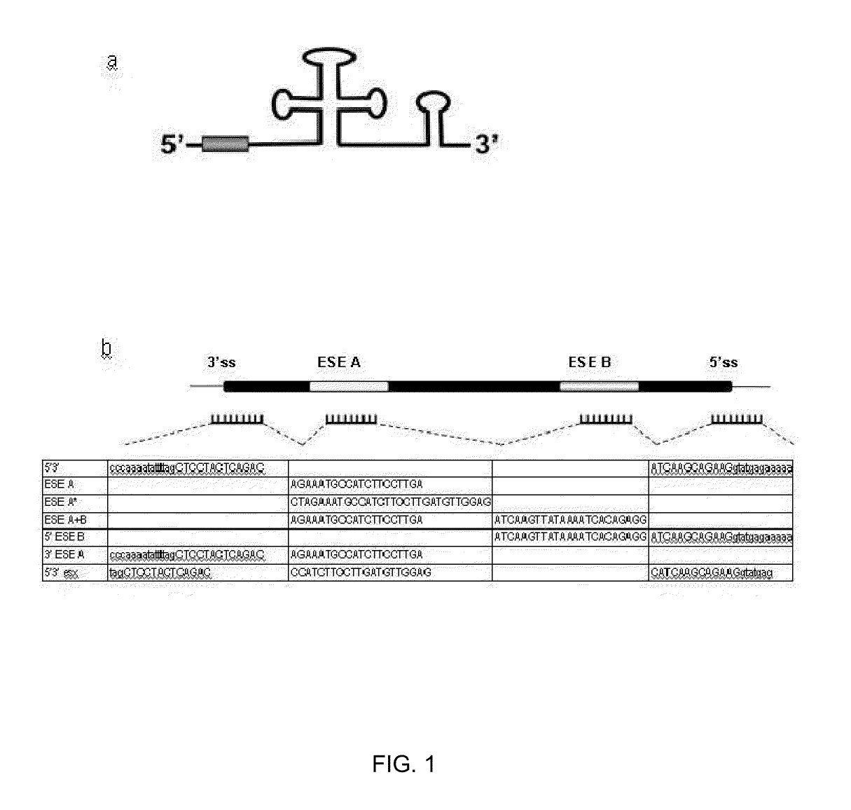

SEQ ID NO: 1 sets out sequence from the 5′ splice junction of exon 51 of the dystrophin pre-mRNA (see also FIG. 1).

SEQ ID NO: 2 sets out sequence from the 3′ splice junction of exon 51 of the dystrophin pre-mRNA (see also FIG. 1).

SEQ ID NO: 3 sets out sequence from an exonic splicing enhancer sequence within exon 51 of the dystrophin gene (see also FIG. 1).

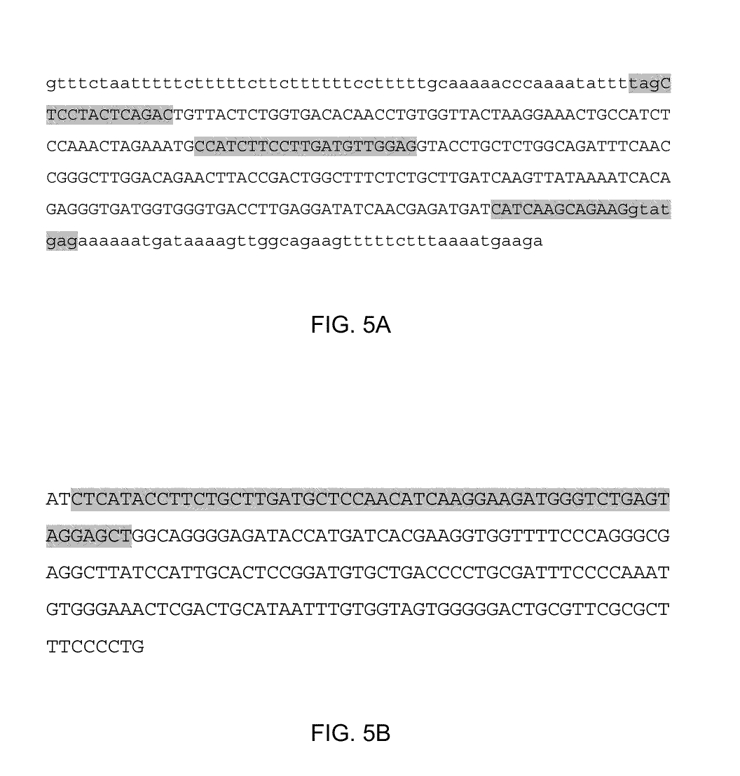

SEQ ID NO: 4 sets out a sequence complementary to the 5′ and 3′ splice junctions and an exonic splicing enhancer of exon 51 of the dystrophin pre-mRNA (see also FIG. 5).

SEQ ID NO: 5 sets out the sequence encoding a modified snRNA (which comprises the sequence set out in SEQ ID NO: 4—see also FIG. 5).

### DETAILED DESCRIPTION OF THE INVENTION

The present invention concerns modified (chimeric) small nuclear RNA (snRNA) molecules carrying antisense sequences targeted against the splice junctions and an exonic splicing enhancer sequence of a pre-mRNA and to nucleic acids encoding such modified snRNA molecules. The invention also relates to vectors, in particular gene therapy vectors, comprising nucleic acids encoding the modified snRNAs and to therapeutic applications of the snRNAs, nucleic acids and vectors of the invention.

In particular, the invention concerns chimeric snRNA molecules able to mask the splice junctions (both 5′ and 3′) and an exonic splicing enhancer sequence of an exon in a pre-mRNA so as to induce “skipping” of the exon (i.e. the exon is spliced out of the primary transcript). This results in a mature mRNA from which the skipped exon is absent. The resulting transcript will then give rise to a shorter protein (than the protein translated from a transcript which includes the relevant exon).

This approach allows skipping of exons which carry mutations resulting in the production of non-functional protein or in the absence of protein (for example if the mutation introduces a frame shift). Skipping of the exon allows a functional, albeit shorter, protein to be produced. Thus, the modified snRNA molecules of the invention may be used in therapy, for example in gene therapy.

Typically, a nucleic acid of the invention is a single molecule which comprises antisense sequence capable of targeting 5′ and 3′ splice site junctions and an exonic splicing enhancer sequence. However, it is possible to provide two or three nucleic acids so that each nucleic acid provides one or two of the antisense sequences. Such nucleic acids may then be provided at the same time to a cell so that skipping of an exon may occur. Three nucleic acids each comprising one of the antisense sequences may be provided or two nucleic acids may be provided, one comprising one of the antisense sequences and the other comprising two of the antisense sequences.

In particular, the modified snRNA molecules of the invention may be used to target the dystrophin pre-RNA for the treatment of muscular dystrophies such as Duchenne Muscular Dystrophy (DMD).

DMD is an X-linked recessive disorder which affects 1 in every 3500 males. It is characterized by the absence of cytoskeletal dystrophin (427 KDa protein) which in turn produces a severe and progressive muscle deterioration. Most DMD mutations consist of deletions and point mutations in the 2.5 Mb dystrophin gene which introduce stop codons and consequently premature translation termination. In a milder form of myopathy, Becker Muscular Dystrophy (BMD), deletions inside the gene produce in-frame mRNAs and consequently shorter but semi-functional dystrophin proteins.

Approaches to the treatment of DMD include the transplantation of normal myoblasts into the muscle tissues lacking this protein, whilst others have tried to restore the correct expression of dystrophin through a gene therapy approach that delivers full-length or mini cDNA copies of dystrophin into cells having the mutated gene. However, several problems still remain to be solved in relation to the latter approach, such as size capacity and transducing activity of the vector and immune response to the “therapeutic” gene.

Another approach is based on the fact that internal in-frame deletions in the dystrophin protein produce only mild myopathic symptoms; therefore, it should be possible, by preventing the inclusion of specific (mutated) exon(s) in the mature dystrophin mRNA, to restore a partially corrected phenotype. In this respect, it is interesting to note that restoration of dystrophin levels as low as 30%, and even significantly less, may be sufficient for substantial therapeutic benefit.

This so-called “exon-specific skipping” approach has been accomplished by the extracellular delivery of synthetic 2′-O-methyl antisense oligonucleotides raised against specific splicing junctions or exonic enhancers. The interaction of the antisense RNA with the corresponding target sequence should mask the utilization of specific splice sites or prevent the binding of enhancer factors during the splicing reaction and determine the skipping of the neighboring mutated exon(s) so as to produce an in-frame dystrophin mRNA, the ultimate effect being the production of a shorter but functional dystrophin protein. A significant drawback to the synthetic oligonucleotide approach is that it is not clear they can be delivered effectively in vivo. In addition, treatment with oligonucleotides requires periodic administrations, i.e. on-going treatment over prolonged periods of time.

To circumvent this problem, herein are described nucleic acids (and vectors comprising the nucleic acids) able to encode chimeric snRNAs (i.e. modified snRNAs) containing antisense sequences which target sequences so as to induce exon skipping.

Large amounts of these modified snRNAs may be expressed in vivo, in a stable and continuous fashion. This strategy has been tested on the human deletion of exons 48-50 (and, in addition, cells from a patient with a deletion of exons 45-50. However, this approach could also be applied to other exons of the dystrophin gene (and indeed to any exon of any gene which it is desired to skip). In the case of dystrophin, the reading frame shift resulting from the deletion is restored to the correct frame by the omission of exon 51 in the mature mRNA; if skipping of this exon is obtained, the result is an mRNA in which exon 47 is linked to exon 52, resulting in an in-frame mRNA.

The antisense sequences in modified snRNAs encoded by nucleic acids according to the invention are designed to pair with specific regions of a cellular pre-mRNA, namely both of the 5′ and 3′ splice junctions and an exonic splicing enhancer. Masking of these regions by the antisense sequences in the modified snRNAs interferes with normal cellular transcript processing, so as to promoter skipping of the targeted exon.

Thus, the inventors have utilized different snRNAs and their corresponding coding sequences in order to express in the nucleus chimeric molecules carrying antisense sequences capable of hybridizing with exon 51 splice junctions and an exonic splicing enhancer sequence and have tested their activity in myoblasts from DMD patients with a deletion of exons 48-50 (or a deletion of exons 45 to 50).

Accordingly, the invention provides a nucleic acid encoding a snRNA. The snRNA is modified so that it comprises antisense sequence(s) capable of hybridizing to (for example complementary to) the 5′ and 3′ splice site junctions and to an exonic splicing enhancer sequence of an exon of a pre-mRNA. The sites are targeted such that the targeted exon sequence is skipped during the splicing process that converts the pre-mRNA into a mature mRNA.

That is to say, according to the invention a nucleic acid is provided that encodes for a modified snRNA; modified so that it contains an antisense sequence(s) capable of hybridizing to a target sequence of a cellular RNA, wherein those target sequences are involved in the maturation or modification of said cellular RNA. The maturation or modification of RNA includes splicing, 3′-end formation, editing etc. and, typically, the cellular RNA encodes for a protein of therapeutic interest.

A nucleic acid according to the invention may be “isolated”. In the context of this invention, an “isolated nucleic acid” or “isolated polynucleotide” is a DNA or RNA, for example, that is not found in nature, i.e. is non-naturally occurring, or one which is not immediately contiguous with one or more of the sequences with which an snRNA encoding sequence is flanked in the naturally occurring genome of the organism from which it is derived. Thus, in one embodiment, an isolated nucleic acid includes some or all of the 5′ non-coding (e.g. promoter) sequences that are immediately contiguous to an snRNA coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment) independent of other sequences. An isolated nucleic acid may be substantially free of cellular material, viral material, or culture medium (when produced by recombinant DNA techniques), or chemical precursors or other chemicals (when chemically synthesized).

As used herein, the terms “nucleic acid molecule” or “polynucleotide” are intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded. The nucleic acid may be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

The antisense sequence(s) (comprised with an snRNA of the invention) is/are capable of hybridizing with, for example is/are complementary to, the two splice junctions (5′ and 3′) and an exonic splicing enhancer sequence of the exon encoding for a protein of therapeutic interest. The sequences targeted within a pre-mRNA are such that the exon sequence is excluded from the maturation process of the pre-mRNA to mature mRNA. Preferably, the antisense sequence(s) comprise(s) sequence complementary to both the 5′ and the 3′ splice junctions and an exonic splicing enhancer sequence of the exon to be excluded.

Small nuclear ribonucleic acid (snRNA) is a class of small RNA molecules that are found within the nucleus of eukaryotic cells. They are transcribed by RNA polymerase II or RNA polymerase III and are involved in a variety of important processes such as RNA splicing (removal of introns from hnRNA), regulation of transcription factors (7SK RNA) or RNA polymerase II (B2 RNA), and maintaining the telomeres. They are always associated with specific proteins, and the complexes are referred to as small nuclear ribonucleoproteins (snRNP). These elements are rich in uridine content. Since snRNAs are localized in the nucleus, they can affect only nuclear processes.

The antisense sequence(s) comprised within the modified snRNAs encoded by a nucleic acid of the invention affect splicing by interacting with two types of sequences:

a) a splice junction (or splice site); and

b) an exonic splicing enhancer.

These two approaches have previously been explored as alternative strategies. Herein, it is shown that a combination of the two approaches, allows the generation of improved snRNA encoding nucleic acids.

Antisense sequences capable of hybridizing with splice sites (both 5′ and 3′) can induce, during splicing, the skipping of a specific exon.

A splice junction or splice site is the boundary between an exon and an intron. There are two varieties: the border going from exon to intron is called a donor site or a 5′ site; the border separating intron from exon is called an acceptor site or a 3′ site. Nearly all splice sites conform to consensus sequences. These consensus sequences include nearly invariant dinucleotides at each end of the intron, GT at the 5′ end of the intron, and AG at the 3′ end of the intron (see FIG. 5a for example). In over 60% of cases, the exon sequence is (A/C)AG at the donor site, and G at the acceptor site.

Accordingly, an antisense sequence capable of hybridizing with to the 5′ and 3′ splice sites will typically be capable of hybridizing with the two exon/intron boundaries of the exon to be skipped with the adjacent. Suitable splice junctions or splice site sequences may readily be identified by the skilled person.

A preferred nucleic acid of the invention is one wherein the sequence which targets the 5′ splice junction of exon 51 of the dystrophin pre-mRNA comprises sequence capable of hybridizing with SEQ ID NO: 1.

A preferred nucleic acid of the invention is one wherein the sequence which targets the 3′ splice junction of exon 51 of the dystrophin pre-mRNA comprises sequence capable of hybridizing with SEQ ID NO: 2.

Sequence capable of hybridizing with SEQ ID NO: 1 or 2 may be complementary to all or part of those sequences.

Many human exons, of large size or with relatively weak flanking splice sites, contain sequences that promote their inclusion in the mRNA, the so called exonic splicing enhancer (ESEs). Such sequences are recognized by serine/arginine (SR)-rich proteins that promote exon utilization in tissues where they are expressed. Typically, an exonic splicing enhancer (ESE) is a DNA sequence motif consisting of 6 bases within an exon that directs, or enhances, accurate splicing of hetero-nuclear RNA or pre-mRNA into mRNA. Again, suitable splice junctions or splice site sequences may readily be identified by the skilled person.

Antisense sequences within the snRNAs of the invention capable of hybridizing with an ESE will typically be capable of hybridizing with such a sequence.

A further preferred nucleic acid according to the invention is one wherein the sequence which targets the exonic splicing enhancer of exon 51 of the dystrophin pre-mRNA comprises sequence capable of hybridizing with SEQ ID NO: 3.

The antisense sequence(s) comprised within a snRNA of the invention is/are capable of hybridizing with the sequences specified above. Such antisense sequence(s) will typically be capable of hybridizing to all or part of the specified target sequence. Generally, therefore the antisense sequence(s) will be complementary to all of or part of such a target sequence. For example, an antisense sequence may be the exact complement of its target sequence. However, absolute complementarity is not required and a preferred antisense sequence is one which has sufficient complementarity (i.e. substantial complementarity) to form a duplex with its target sequence having a melting temperature of greater than 40° C. under physiological conditions.

Alternatively, an antisense sequence will have sufficient complementarity such that it will hybridize with the target sequence such that the target sequence cannot interact with the splicing machinery in the way that it would in the absence of the antisense sequence. Obscuring the target sequence in this way allows exon skipping to occur.

The antisense sequence may be an antisense sequence which hybridises to the specified target sequence under conditions of medium to high stringency, such as 0.03M sodium chloride and 0.03M sodium citrate at from about 45° C. to about 65° C.

Alternatively, stringent hybridisation conditions may be defined as conditions that allow a nucleic acid sequence of at least about 15 nucleotides in length to hybridise at a temperature of about 65° C. in a solution comprising about 1 M salt, preferably 6×SSC (sodium chloride, sodium citrate) or any other solution having a comparable ionic strength, and washing at 65° C. in a solution comprising about 0.1 M salt, or less, preferably 0.2×SSC or any other solution having a comparable ionic strength. These conditions may allow the specific hybridisation of sequences having about 90% or more sequence identity.

Moderate conditions may be defined as conditions that allow a nucleic acid sequences of at least 15 nucleotides in length to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. These conditions will usually allow the specific hybridisation of sequences having up to 50% sequence identity. The person skilled in the art will be able to modify these hybridisation conditions in order to specifically identify sequences varying in identity between 50% and 90%.

An antisense sequence may be defined with reference to a specific sequence identity to the reverse complement of the sequence to which it is intended to target. Thus, a modified snRNA of the invention will comprise antisense sequences having at least about 70% sequence identity with the reverse complements of the 5′ and 3′ splice junction sequences and an exonic splicing enhancer sequence of the target exon (for example the reverse complements of SEQ ID NOs: 1, 2 or 3). The antisense sequences will typically have at least about 75%, preferably at least about 80%, at least about 85%, at least about 90%, at least about 92%, at least about 93%, at least about 94%, at least about 95%, at least about 96%, at least about 97%, at least about 98% or at least about 99% or at least 75%, at least 80%, at least 85%, at least 90%, at least 92%, at least 93%, at least 94%, at least 95%, at least 96%, at least 97%, at least 98% or at least 99% sequence identity with the reverse complements of their target sequences (i.e. the reverse complements of the 5′ and 3′ splice junctions and an exonic splicing enhancer of the target exon, for example the reverse complements of SEQ ID NOs: 1, 2 or 3).

In a nucleic acid of the invention the portion encoding the antisense sequence (targeting the 5′ and 3′ splice sites and an exonic splicing enhancer of an exon) may have at least about 75%, preferably at least about 80%, at least about 85%, at least about 90%, at least about 92%, at least about 93%, at least about 94%, at least about 95%, at least about 96%, at least about 97%, at least about 98% or at least about 99% or at least 75%, at least 80%, at least 85%, at least 90%, at least 92%, at least 93%, at least 94%, at least 95%, at least 96%, at least 97%, at least 98% or at least 99% sequence identity with the sequence set out in SEQ ID NO: 4.

Sequence identity (or sequence similarity) is herein defined as a relationship between two or more nucleic acid (polynucleotide) sequences, as determined by comparing the sequences. Usually, sequence identities or similarities are compared, typically over the whole length of the sequences compared. However, sequences may be compared over shorter comparison windows. In the art, “identity” also means the degree of relatedness between nucleic acid sequences, as the case may be, as determined by the match between strings of such sequences.

Preferred methods to determine identity are designed to give the largest match between the sequences tested. Methods to determine identity and similarity are codified in publicly available computer programs. Preferred computer program methods to determine identity and similarity between two sequences include e.g. the BestFit, BLASTP, BLASTN, and FASTA (Altschul, S. F. et al., J. MoI. Biol. 215:403-410 (1990), publicly available from NCBI and other sources. Preferred parameters for nucleic acid sequences comparison using BLASTP are gap open 1 1.0, gap extend 1, DNA full matrix (DNA identity matrix).

The results described herein demonstrate that the combination of antisense sequences targeting the 5′ and 3′ splice junction of an exon and an exonic splicing enhancer sequence of that exon can induce efficient skipping of an exon and rescue of protein synthesis.

Targeting of splice sites and an exonic splicing enhancer can be applied in all those cases where the skipping of a specific mutated exon can restore the functionality of the mRNA (such as in the case of dystrophin mutations). That is to say, a nucleic acid of the invention is typically such that skipping of the targeted exon by the modified snRNA leads to the production of a functional protein.

Since more than half of human genes encode transcripts that undergo alternative splicing, the nucleic acid of the invention provides a general method for control of the expression of alternatively spliced isoforms from specific genes and may thus be useful in a wide variety of applications. The snRNAs encoded by nucleic acids of the invention can be used to control the type of alternative spliced mRNA produced and of the corresponding protein.

A nucleic acid of the invention may be such that the exon to be skipped is an exon of the dystrophin pre-mRNA. That is to say, in a preferred aspect, the protein of therapeutic interest containing an exon to be deleted is dystrophin. Preferably, the exclusion of the mutated exon leads to the production of a functional dystrophin protein (the exon being mutated so as to contain or to determine the production of a premature stop and/or frame shift). In particular, the nucleic acid of the invention may be such that exon 51 of the dystrophin pre-mRNA is skipped.

A nucleic acid of the invention comprises sequence(s) capable of hybridizing with, for example complementary to, the 5′ and 3′ splice site junctions and to an exonic splicing enhancer of an exon. The sequence complementary to the 5′ and 3′ splice site junctions and to an exonic splicing enchancer of an exon may be comprised with the 5′ region of the snRNA. The antisense sequences may be the most 5′ sequences in the snRNA or may be situated at least one, two, three, four, five, six, seven, eight, nine or ten or more nucleotides 3′ from the 5′ end of the modified snRNA.

The antisense sequences of a modified snRNA may modify the snRNA by way of addition and/or by way of substitution. That is to say, the antisense sequences may simply be added to a snRNA or, alternatively, may replace sequence(s) within a snRNA. If the antisense sequences are added by way of substitution, the modified snRNA may be of the same length as a wild-type snRNA. Of course, the sequences replaced by the antisense sequences may be shorter or longer than the antisense sequences.

The antisense sequence(s) complementary to the 5′ and 3′ splice site junctions and to an exonic splicing enhancer of an exon may be arranged adjacent to each other in a modified snRNA of the invention (i.e. placed adjacent to each other with no intervening nucleotides). Alternatively the antisense sequences may be separated by one or more nucleotides, for example by one, two, three, four, five, six, seven, eight, nine or ten or more nucleotides. Two of the antisense sequences may be placed adjacent to one another (i.e. no intervening nucleotides) with the third antisense sequence separated from those two by one or more intervening nucleotides as just described.

Thus, an additional preferred nucleic acid of the invention is one wherein the sequence complementary to the 5′ and 3′ splice junctions and an exonic splicing enhancer of exon 51 of the dystrophin pre-mRNA comprises the sequence set out in SEQ ID NO: 4.

A nucleic acid of the invention may preferably comprise the sequence set out in SEQ ID NO: 5.

A nucleic acid of the invention may encode a modified snRNA which is a modified U1, U2, U3, U4, U5, U6 or U7 snRNA. Preferably, the nucleic acid encodes for a modified U1, U2 or U7 snRNA.

If the snRNA is based on a U1 snRNA, it is typically modified such that a 5′ fragment of the nucleic acid encoding a modified snRNA is replaced by the antisense sequences described herein.

If the snRNA is based on a U2 snRNA, it is typically modified such that the U2 region complementary to the intron branch site is replaced by the antisense sequences described herein.

In an alternative preferred aspect, a U2 based snRNA is further modified such that it does not interact with U6 snRNA, for example whilst maintaining its secondary and tertiary structures.

The modified snRNA encoded by a nucleic acid of the invention, in particular in the case of a U1 snRNA, may retain the ability to interact with the U1 associated 70K protein.

The invention provides a modified snRNA encoded by a nucleic acid according to any one of the preceding claims.

The invention also provides a vector which comprises a nucleic acid of the invention. That is to say, a nucleic acid encoding a modified snRNA as described herein may be expressed in a cell from a suitable vector. A suitable vector is typically a recombinant replicable vector comprising a sequence which, when transcribed, gives rise to a modified snRNA of the invention.

Typically, the sequence encoding the polynucleotide is operably linked to a control sequence which is capable of providing for the transcription of the sequence giving rise to a modified snRNA. The term “operably linked” refers to a juxtaposition wherein the components described are in a relationship permitting them to function in their intended manner. A control sequence “operably linked” to a sequence giving rise to an snRNA is ligated in such a way that transcription of the sequence is achieved under conditions compatible with the control sequences.

A vector for use in the invention may be for example, a plasmid or a virus vector. It will be understood by those skilled in the art that the nucleic acid of the invention may be operably linked with appropriate control sequences. For example, the nucleic of the invention may be operably associated with expression control elements, such as transcription/translation control signals, origins of replication, polyadenylation signals, and internal ribosome entry sites (IRES), promoters, regulators of a promoter (such as an enhancer), and the like.

A vector may contain one or more selectable marker genes, for example an ampicillin resistance gene in the case of bacterial plasmid or a neomycin resistance gene for a mammalian vector.

A vector of the invention may be used in vitro or ex vivo, for example for the production of antisense RNA, or used to transfect or transform a host cell. The vector may also be suitable for or adapted for use in vivo, for example in a method of gene therapy.

Promoters/enhancers and other expression regulation signals may be selected to be compatible with the host cell for which the vector is designed.

In a vector of the invention, at least one nucleic acid sequence encoding a modified snRNA for expression in a mammalian cell, preferably is/are operably linked to at least one mammalian cell-compatible expression control sequence, e.g., a promoter. The promoter used may be one which typically drives expression of the snRNA on which the modified snRNA of the invention is based.

Many such promoters are known in the art (see Sambrook and Russel, 2001, supra). Constitutive promoters that are broadly expressed in many cell-types, such as the CMV promoter or b-actin promoter may be used. However, more preferred will be promoters that are inducible, tissue-specific, cell-type-specific, or cell cycle-specific. For example, for liver-specific expression a promoter may be selected from an α1-anti-trypsin promoter, a thyroid hormone-binding globulin promoter, an albumin promoter, LPS (thyroxine-binding globlin) promoter, HCR-ApoCII hybrid promoter, HCR-hAAT hybrid promoter and an apolipoprotein E promoter. Other examples include the E2F promoter for tumor-selective, and, in particular, neurological cell tumor-selective expression (Parr et al., 1997, Nat. Med. 3:1145-9) or the IL-2 promoter for use in mononuclear blood cells (Hagenbaugh et al., 1997, J Exp Med; 185: 2101-10).

Viral promoters may also be used, for example the Moloney murine leukaemia virus long terminal repeat (MMLV LTR), the promoter rous sarcoma virus (RSV) LTR promoter, the SV40 promoter, the human cytomegalovirus (CMV) IE promoter, herpes simplex virus promoters, adenovirus promoters or adeno-associated virus promoters. All of these promoters are readily available in the art. Preferred promoters may be tissue specific promoters, for example promoters driving expression specifically within muscle cells.

Those skilled in the art will appreciate that a variety of promoter/enhancer elements may be used depending on the level and tissue specific expression desired. The promoter/enhancer may be constitutive or inducible, depending on the pattern of expression desired. The promoter/enhancer may be native or foreign and can be a natural or a synthetic sequence. By foreign, it is intended that the transcriptional initiation region is not found in the wild-type host into which the transcriptional initiation region is introduced.

Promoter/enhancer elements that are native to the target cell or subject to be treated are most preferred. Also preferred are promoters/enhancer elements that are native to the nucleic acid of the invention. The promoter/enhancer element is chosen so that it will function in the target cell(s) of interest. Mammalian promoter/enhancer elements are typically preferred. The promoter/enhancer element may be constitutive or inducible.

A vector of the invention may further include additional sequences, for example additional sequences flanking the sequence giving rise to the modified snRNA. For example, a vector may comprise sequences homologous to eukaryotic genomic sequences, preferably mammalian genomic sequences, or viral genomic sequences. This will allow the introduction of a nucleic acid of the invention into the genome of a eukaryotic cell or a virus by homologous recombination.

Such a vector will typically allow stable and efficient expression in a target cell of one or more nucleic acids according to the invention (i.e. the expression of one or more modified snRNAs as described herein).

The target cell may be any desired cell type, such as a muscle cell, for example a fiber cell or a satellite muscle cell. A vector of the invention may be suitable for transfer to a stem cell.

In order to obtain effective “therapeutic” snRNA molecules in vivo, several parameters may need to be considered. Not only must a nucleic acid (encoding a modified snRNA) be operably linked to an efficient promoter capable of producing a high level of expression but, in addition, the RNA context in which the therapeutic RNA is embedded should provide stability and/or specific subcellular localization.

The last point may be crucial since, in the cell, RNAs are sorted to specific cellular locations (nucleus, nucleolus, cytoplasm, free and polysome-bound RNPs, etc.) and efficient activity of the therapeutic molecule can be obtained only if co-localization with the target is ensured.

Small cellular RNAs have been advantageously used as vectors since they are normally transcribed from strong promoters (Pol-I or Pol-II) and because they allow the delivery of the chimeric constructs into specific cellular compartments.

A vector according to the invention may comprise two or more, for example three, four or five or more nucleic acids of the invention, each encoding a modified snRNA. Each snRNA may be designed to target a different exon within the same gene or may be designed to target exons within different genes (or a combination thereof).

A vector according to the invention may be a gene delivery vector. Such a gene delivery vector may be a viral gene delivery vector or a non-viral gene delivery vector.

Non-viral gene delivery may be carried out using naked DNA which is the simplest method of non-viral transfection. It may be possible, for example, to administer a nucleic acid of the invention using naked plasmid DNA. Alternatively, methods such as electroporation, sonoporation or the use of a “gene gun”, which shoots DNA coated gold particles into the cell using, for example, high pressure gas or an inverted .22 caliber gun, may be used.

To improve the delivery of a nucleic acid into the cell, it may be necessary to protect it from damage and its entry into the cell may be facilitated. To this end, lipoplexes and polyplexes may be used that have the ability to protect a nucleic acid from undesirable degradation during the transfection process.

Plasmid DNA may be coated with lipids in an organized structure such as a micelle or a liposome. When the organized structure is complexed with DNA it is called a lipoplex. Anionic and neutral lipids may be used for the construction of lipoplexes for synthetic vectors. Preferably, however, cationic lipids, due to their positive charge, may be used to condense negatively charged DNA molecules so as to facilitate the encapsulation of DNA into liposomes. If may be necessary to add helper lipids (usually electroneutral lipids, such as DOPE) to cationic lipids so as to form lipoplexes.

Complexes of polymers with DNA, called polyplexes, may be used to deliver a nucleic acid of the invention. Most polyplexes consist of cationic polymers and their production is regulated by ionic interactions. Polyplexes typically cannot release their DNA load into the cytoplasm. Thus, co-transfection with endosome-lytic agents (to lyse the endosome that is made during endocytosis, the process by which the polyplex enters the cell), such as inactivated adenovirus, may be necessary.

Hybrid methods may be used to deliver a nucleic acid of the invention that combines two or more techniques. Virosomes are one example; they combine liposomes with an inactivated HIV or influenza virus. Other methods involve mixing other viral vectors with cationic lipids or hybridizing viruses and may be used to deliver a nucleic acid of the invention.

A dendrimer may be used to deliver a nucleic acid of the invention, in particular, a cationic dendrimer, i.e. one with a positive surface charge. When in the presence of genetic material such as DNA or RNA, charge complimentarity leads to a temporary association of the nucleic acid with the cationic dendrimer. On reaching its destination the dendrimer-nucleic acid complex is then taken into the cell via endocytosis.

More typically, a suitable viral gene delivery vector may be used to deliver a nucleic acid of the invention. Viral vectors suitable for use in the invention may be a parvovirus, an adenovirus, a retrovirus, a lentivirus or a herpes simplex virus. The parvovirus may be an adenovirus-associated virus (AAV).

As used herein, in the context of gene delivery, the term “vector” or “gene delivery vector” may refer to a particle that functions as a gene delivery vehicle, and which comprises nucleic acid (i.e., the vector genome) packaged within, for example, an envelope or capsid. Alternatively, in some contexts, the term “vector” may be used to refer only to the vector genome.

Accordingly, the present invention provides gene delivery vectors (comprising a nucleic acid of the invention) based on animal parvoviruses, in particular dependoviruses such as infectious human or simian AAV, and the components thereof (e.g., an animal parvovirus genome) for use as vectors for introduction and/or expression of a modified snRNA in a mammalian cell.

Viruses of the Parvoviridae family are small DNA animal viruses. The family Parvoviridae may be divided between two subfamilies: the Parvovirinae, which infect vertebrates, and the Densovirinae, which infect insects. Members of the subfamily Parvovirinae are herein referred to as the parvoviruses and include the genus Dependovirus. As may be deduced from the name of their genus, members of the Dependovirus are unique in that they usually require coinfection with a helper virus such as adenovirus or herpes virus for productive infection in cell culture. The genus Dependovirus includes AAV, which normally infects humans (e.g., serotypes 1, 2, 3A, 3B, 4, 5, and 6) or primates (e.g., serotypes 1 and 4), and related viruses that infect other warm-blooded animals (e.g., bovine, canine, equine, and ovine adeno-associated viruses). Further information on parvoviruses and other members of the Parvoviridae is described in Kenneth I. Berns, “Parvoviridae: The Viruses and Their Replication,” Chapter 69 in Fields Virology (3d Ed. 1996). For convenience the present invention is further exemplified and described herein by reference to AAV. It is, however, understood that the invention is not limited to AAV but may equally be applied to other parvoviruses.

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, -2 and -3) form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. Following wtAAV infection in mammalian cells the Rep genes (i.e. Rep78 and Rep52) are expressed from the P5 promoter and the P19 promotor, respectively and both Rep proteins have a function in the replication of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

In an AAV suitable for use as a gene therapy vector, the vector genome typically comprises a nucleic acid of the invention (as described herein) to be packaged for delivery to a target cell. According to this particular embodiment, the heterologous nucleotide sequence is located between the viral ITRs at either end of the vector genome. In further preferred embodiments, the parvovirus (e.g. AAV) cap genes and parvovirus (e.g. AAV) rep genes are deleted from the template genome (and thus from the virion DNA produced therefrom). This configuration maximizes the size of the nucleic acid sequence(s) that can be carried by the parvovirus capsid.

According to this particular embodiment, the nucleic acid of the invention is located between the viral ITRs at either end of the substrate. It is possible for a parvoviral genome to function with only one ITR. Thus, in a gene therapy vector of the invention based on a parvovirus, the vector genome is flanked by at least one ITR, but, more typically, by two AAV ITRs (generally with one either side of the vector genome, i.e. one at the 5′ end and one at the 3′ end). There may be intervening sequences between the nucleic acid of the invention in the vector genome and one or more of the ITRs.

Preferably, the nucleic acid encoding a modified snRNA (for expression in the mammalian cell) will be incorporated into a parvoviral genome located between two regular ITRs or located on either side of an ITR engineered with two D regions.

AAV sequences that may be used in the present invention for the production of AAV gene therapy vectors can be derived from the genome of any AAV serotype. Generally, the AAV serotypes have genomic sequences of significant homology at the amino acid and the nucleic acid levels, provide an identical set of genetic functions, produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71: 6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74: 8635-47). AAV serotype 1, 2, 3, 4, 5, 6, 7, 8 or 9 may be used in the present invention. However, AAV serotypes 1 and 6 are preferred sources of AAV sequences for use in the context of the present invention.

Preferably the AAV ITR sequences for use in the context of the present invention are derived from AAV1, AAV2, AAV4 and/or AAV6. Likewise, the Rep (Rep78 and Rep52) coding sequences are preferably derived from AAV1, AAV2, AAV4 and/or AAV6. The sequences coding for the VP1, VP2, and VP3 capsid proteins for use in the context of the present invention may however be taken from any of the known 42 serotypes, more preferably from AAV1, AAV2, AAV3, AAV4, AAV5, AAV6, AAV7, AAV8 or AAV9 or newly developed AAV-like particles obtained by e.g. capsid shuffling techniques and AAV capsid libraries.

AAV Rep and ITR sequences are particularly conserved among most serotypes. The Rep78 proteins of various AAV serotypes are e.g. more than 89% identical and the total nucleotide sequence identity at the genome level between AAV2, AAV3A, AAV3B, and AAV6 is around 82% (Bantel-Schaal et al., 1999, J. Virol., 73 (2):939-947). Moreover, the Rep sequences and ITRs of many AAV serotypes are known to efficiently cross-complement (i.e., functionally substitute) corresponding sequences from other serotypes in production of AAV particles in mammalian cells. US2003148506 reports that AAV Rep and ITR sequences also efficiently cross-complement other AAV Rep and ITR sequences in insect cells.

The AAV VP proteins are known to determine the cellular tropicity of the AAV virion. The VP protein-encoding sequences are significantly less conserved than Rep proteins and genes among different AAV serotypes. The ability of Rep and ITR sequences to cross-complement corresponding sequences of other serotypes allows for the production of pseudotyped AAV particles comprising the capsid proteins of a serotype (e.g., AAV1 or 6) and the Rep and/or ITR sequences of another AAV serotype (e.g., AAV2). Such pseudotyped rAAV particles are a part of the present invention.

Modified “AAV” sequences also can be used in the context of the present invention, e.g. for the production of AAV gene therapy vectors. Such modified sequences e.g. include sequences having at least about 70%, at least about 75%, at least about 80%, at least about 85%, at least about 90%, at least about 95%, or more nucleotide and/or amino acid sequence identity (e.g., a sequence having about 75-99% nucleotide sequence identity) to an AAV1, AAV2, AAV3, AAV4, AAV5, AAV6, AAV7, AAV8 or AAV9 ITR, Rep, or VP can be used in place of wild-type AAV ITR, Rep, or VP sequences.

Although similar to other AAV serotypes in many respects, AAV5 differs from other human and simian AAV serotypes more than other known human and simian serotypes. In view thereof, the production of rAAV5 can differ from production of other serotypes in insect cells. Where methods of the invention are employed to produce rAAV5, it is preferred that one or more constructs comprising, collectively in the case of more than one construct, a nucleotide sequence comprising an AAV5 ITR, a nucleotide sequence comprises an AAV5 Rep coding sequence (i.e. a nucleotide sequence comprises an AAV5 Rep78). Such ITR and Rep sequences can be modified as desired to obtain efficient production of AAV5 or pseudotyped AAV5 vectors. E.g., the start codon of the Rep sequences can be modified, VP splice sites can be modified or eliminated, and/or the VP1 start codon and nearby nucleotides can be modified to improve the production of AAV5 vectors.

Thus, the viral capsid used in the invention may be from any parvovirus, either an autonomous parvovirus or dependovirus, as described above. Preferably, the viral capsid is an AAV capsid (e.g., AAV1, AAV2, AAV3, AAV4, AAV5 or AAV6 capsid). In general, the AAV1 capsid or AAV6 capsid are preferred. The choice of parvovirus capsid may be based on a number of considerations as known in the art, e.g., the target cell type, the desired level of expression, the nature of the heterologous nucleotide sequence to be expressed, issues related to viral production, and the like. For example, the AAV1 and AAV6 capsid may be advantageously employed for skeletal muscle; AAV1, AAV5 and AAV8 for the liver and cells of the central nervous system (e.g., brain); AAV5 for cells in the airway and lung or brain; AAV3 for bone marrow cells; and AAV4 for particular cells in the brain (e.g., appendable cells).

It is within the technical skills of the skilled person to select the most appropriate virus or virus subtype. Some subtypes may be more appropriate than others for a certain type of tissue.

For example, muscle-specific expression of a modified snRNA of the invention intended for the skipping of an exon of the dystrophin gene may advantageously be induced by AAV-mediated transduction of muscle cells. Muscle is amenable to AAV-mediated transduction, and different serotypes may be used (AAV1, AAV6, AAV7, AAVB). Transduction of muscle may be accomplished by intramuscular injection of an AAV encoding an snRNA of the invention in multiple sites. However, intravenous it intra-arterial administration is also be applicable (AAV1, AAV6, AAVB) and may be more suitable to achieve administration to all muscle groups.

A parvovirus gene therapy vector prepared according to the invention may be a “hybrid” particle in which the viral TRs and viral capsid are from different parvoviruses. Preferably, the viral TRs and capsid are from different serotypes of AAV.

Likewise, the parvovirus may have a “chimeric” capsid (e.g., containing sequences from different parvoviruses, preferably different AAV serotypes) or a “targeted” capsid (e.g., a directed tropism).

In the context of the invention “at least one parvoviral ITR nucleotide sequence” is understood to mean a palindromic sequence, comprising mostly complementary, symmetrically arranged sequences also referred to as “A,” “B,” and “C” regions. The ITR functions as an origin of replication, a site having a “cis” role in replication, i.e., being a recognition site for trans acting replication proteins such as e.g. Rep 78 (or Rep68) which recognize the palindrome and specific sequences internal to the palindrome. One exception to the symmetry of the ITR sequence is the “D” region of the ITR. It is unique (not having a complement within one ITR). Nicking of single-stranded DNA occurs at the junction between the A and D regions. It is the region where new DNA synthesis initiates. The D region normally sits to one side of the palindrome and provides directionality to the nucleic acid replication step. An parvovirus replicating in a mammalian cell typically has two ITR sequences. It is, however, possible to engineer an ITR so that binding sites are on both strands of the A regions and D regions are located symmetrically, one on each side of the palindrome. On a double-stranded circular DNA template (e.g., a plasmid), the Rep78- or Rep68-assisted nucleic acid replication then proceeds in both directions and a single ITR suffices for parvoviral replication of a circular vector. Thus, one ITR nucleotide sequence can be used in the context of the present invention. Preferably, however, two or another even number of regular ITRs are used. Most preferably, two ITR sequences are used. A preferred parvoviral ITR is an AAV ITR. For safety reasons it may be desirable to construct a parvoviral (AAV) vector that is unable to further propagate after initial introduction into a cell. Such a safety mechanism for limiting undesirable vector propagation in a recipient may be provided by using AAV with a chimeric ITR as described in US2003148506.

Those skilled in the art will appreciate that the viral Rep protein(s) used for producing an AAV vector of the invention may be selected with consideration for the source of the viral ITRs. For example, the AAV5 ITR typically interacts more efficiently with the AAV5 Rep protein, although it is not necessary that the serotype of ITR an Rep protein(s) are matched.

The ITR(s) used in the invention are typically functional, i.e. they may be fully resolvable and are preferably AAV sequences, with serotypes 1, 2, 3, 4, 5 or 6 being preferred. Resolvable AAV ITRs according to the present invention need not have a wild-type ITR sequence (e.g., a wild-type sequence may be altered by insertion, deletion, truncation or missense mutations), as long as the ITR mediates the desired functions, e.g., virus packaging, integration, and/or provirus rescue, and the like.

Advantageously, use of a gene therapy vector as compared with previous approaches that uses antisense oligonucleotides, the skipping of the mutated exon and the restoration of protein synthesis (for example dystrophin synthesis) are characteristics that the transduced cells acquire permanently, thus avoiding the need for continuous administration to achieve a therapeutic effect.

Accordingly, the vectors of the invention therefore represent a tool for the development of strategies for the in vivo delivery of therapeutic snRNAs, by engineering the snRNA encoding within a gene therapy vector that efficiently transduces an appropriate cell type, such as muscle fiber cells.

The invention provides a host cell, such as a mammalian or insect cell, comprising a nucleic acid, a modified snRNA or a vector according to the invention. A nucleic acid, modified snRNA or vector of the invention may be introduced into a suitable host cell by any appropriate transformation or transfection technique.

Preferably, the host cell will permit the expression of the modified snRNA. Thus, the host cell may be, for example, a bacterial, a yeast, an insect or a mammalian cell.

Any insect cell which allows for replication of a recombinant parvoviral (rAAV) vector and which can be maintained in culture can be used in accordance with the present invention. For example, the cell line used can be from Spodoptera frugiperda, drosophila cell lines, or mosquito cell lines, e.g., Aedes albopictus derived cell lines. Preferred insect cells or cell lines are cells from the insect species which are susceptible to baculovirus infection, including e.g. Se301, SeIZD2109, SeUCR1, Sf9, Sf900+, Sf21, BTI-TN-5B1-4, MG-1, Tn368, HzAm1, Ha2302, Hz2E5, High Five (Invitrogen, CA, USA) and expresSF+® (U.S. Pat. No. 6,103,526; Protein Sciences Corp., CT, USA).

Mammalian cells suitable for use in the invention may be of human or non-human origin.

In addition, the invention provides a method for the preparation of a parvoviral gene delivery vector which method comprising the steps of:
(a) providing an insect cell comprising one or more nucleic acid constructs comprising:
   (i) a nucleic acid of the invention that is flanked by at least one parvoviral inverted terminal repeat nucleotide sequence;
   (ii) a first expression cassette comprising a nucleotide sequence encoding one or more parvoviral Rep proteins which is operably linked to a promoter that is capable of driving expression of the Rep protein(s) in the insect cell;
   (iii) a second expression cassette comprising a nucleotide sequence encoding one or more parvoviral capsid proteins which is operably linked to a promoter that is capable of driving expression of the capsid protein(s) in the insect cell;
(b) culturing the insect cell defined in (a) under conditions conducive to the expression of the Rep and the capsid proteins; and, optionally,
(c) recovery of the parvoviral gene delivery vector.

In general, therefore, the method of the invention allows the production of a parvoviral gene delivery vector (comprising a nucleic acid of the invention) in an insect cell. Preferably, the method comprises the steps of: (a) culturing an insect cell as defined above under conditions such that the parvoviral (eg. AAV) vector is produced; and, (b) recovery of the recombinant parvoviral (eg. AAV) vector.

It is understood here that the (AAV) vector produced in such a method preferably is an infectious parvoviral or AAV virion that comprises a parvoviral genome, which itself comprises a nucleic acid of the invention. Growing conditions for insect cells in culture, and production of heterologous products in insect cells in culture are well-known in the art and described e.g. in the above cited references on molecular engineering of insects cells.

In a method of the invention, a nucleic acid of the invention that is flanked by at least one parvoviral ITR sequence is provided. This type of sequence is described in detail above. Preferably, the nucleic acid of the invention is sequence is located between two parvoviral ITR sequences.

The first expression cassette comprises a nucleotide sequence encoding one or more parvoviral Rep proteins which is operably linked to a first promoter that is capable of driving expression of the Rep protein(s) in the insect cell.

A nucleotide sequence encoding animal parvoviruses Rep proteins, is herein understood as a nucleotide sequence encoding the non-structural Rep proteins that are required and sufficient for parvoviral vector production in insect cells such the Rep78 and Rep52 proteins, or the Rep68 and Rep40 proteins, or the combination of two or more thereof.

The animal parvovirus nucleotide sequence preferably is from a dependovirus, more preferably from a human or simian adeno-associated virus (AAV) and most preferably from an AAV which normally infects humans (e.g., serotypes 1, 2, 3A, 3B, 4, 5, and 6) or primates (e.g., serotypes 1 and 4). Rep coding sequences are well known to those skilled in the art and suitable sequences are referred to and described in detail in WO2007/148971 and also in WO2009/014445.

Preferably, the nucleotide sequence encodes animal parvoviruses Rep proteins that are required and sufficient for parvoviral vector production in insect cells.

The second expression cassette comprises a nucleotide sequence encoding one or more parvoviral capsid proteins which is operably linked to a promoter that is capable of driving expression of the capsid protein(s) in the insect cell. The capsid protein(s) expressed may be one or more of those described above.

Preferably, the nucleotide sequence encodes animal parvoviruses cap proteins that are required and sufficient for parvoviral vector production in insect cells.

These three sequences (genome, rep encoding and cap encoding) are provided in an insect cell by way of one or more nucleic acid constructs, for example one, two or three nucleic acid constructs. Preferably then, the one or nucleic acid constructs for the vector genome and expression of the parvoviral Rep and cap proteins in insect cells is an insect cell-compatible vector. An “insect cell-compatible vector” or “vector” is understood to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. In a preferred embodiment, the vector is a baculovirus, a viral vector, or a plasmid. In a more preferred embodiment, the vector is a baculovirus, i.e. the construct is a baculoviral vector. Baculoviral vectors and methods for their use are well known to those skilled in the art.

Typically then, a method of the invention for producing a parvoviral gene delivery vector comprises: providing to a cell permissive for parvovirus replication (a) a nucleotide sequence encoding a template for producing vector genome of the invention (as described in detail herein); (b) nucleotide sequences sufficient for replication of the template to produce a vector genome (the first expression cassette defined above); (c) nucleotide sequences sufficient to package the vector genome into a parvovirus capsid (the second expression cassette defined above), under conditions sufficient for replication and packaging of the vector genome into the parvovirus capsid, whereby parvovirus particles comprising the vector genome encapsidated within the parvovirus capsid are produced in the cell. Preferably, the parvovirus replication and/or capsid coding sequences are AAV sequences.

A method of the invention may preferably comprise the step of affinity-purification of the (virions comprising the) recombinant parvoviral (rAAV) vector using an anti-AAV antibody, preferably an immobilised antibody. The anti-AAV antibody preferably is an monoclonal antibody. A particularly suitable antibody is a single chain camelid antibody or a fragment thereof as e.g. obtainable from camels or llamas (see e.g. Muyldermans, 2001, Biotechnol. 74: 277-302). The antibody for affinity-purification of rAAV preferably is an antibody that specifically binds an epitope on a AAV capsid protein, whereby preferably the epitope is an epitope that is present on capsid protein of more than one AAV serotype. E.g. the antibody may be raised or selected on the basis of specific binding to AAV2 capsid but at the same time also it may also specifically bind to AAV1, AAV3, AAV5, AAV6 or AAV8 capsids.

The invention also provides a means for delivering a nucleic acid of the invention into a broad range of cells, including dividing and non-dividing cells. The present invention may be employed to deliver a nucleic acid of the invention to a cell in vitro, e.g. to produce a modified snRNA in vitro or for ex vivo gene therapy.

The cells, pharmaceutical formulations, and methods of the present invention are additionally useful in a method of delivering a nucleic acid of the invention to a host in need thereof, i.e., a nucleic acid encoding a modified snRNA to as to induce skipping of an exon. In this manner, a functional version of a target polypeptide may thus be produced in vivo in the subject.

The present invention finds use in both veterinary and medical applications. Suitable subjects for gene delivery methods as described herein include both avians and mammals, with mammals being preferred. The term “avian” as used herein includes, but is not limited to, chickens, ducks, geese, quail, turkeys and pheasants. The term “mammal” as used herein includes, but is not limited to, humans, bovines, ovines, caprines, equines, felines, canines, lagomorphs, etc. Human subjects are most preferred. Human subjects include neonates, infants, juveniles, and adults.

The invention thus provides a pharmaceutical composition comprising a nucleic acid, a modified snRNA or a vector of the invention and a pharmaceutically acceptable carrier or diluent and/or other medicinal agent, pharmaceutical agent or adjuvant, etc.

For injection, the carrier will typically be a liquid. For other methods of administration, the carrier may be either solid or liquid. For inhalation administration, the carrier will be respirable, and will preferably be in solid or liquid particulate form. As an injection medium, it is preferred to use water that contains the additives usual for injection solutions, such as stabilizing agents, salts or saline, and/or buffers.

In general, a “pharmaceutically acceptable carrier” is one that is not toxic or unduly detrimental to cells. Exemplary pharmaceutically acceptable carriers include sterile, pyrogen-free water and sterile, pyrogen-free, phosphate buffered saline. Pharmaceutically acceptable carriers include physiologically acceptable carriers. The term “pharmaceutically acceptable carrier” includes any and all solvents, dispersion media, coatings, antibacterial and antifungal agents, isotonic and absorption delaying agents, and the like that are physiologically compatible”.

By “pharmaceutically acceptable” it is meant a material that is not biologically or otherwise undesirable, i.e, the material may be administered to a subject without causing any undesirable biological effects. Thus, such a pharmaceutical composition may be used, for example, in transfection of a cell ex vivo or in administering a viral particle or cell directly to a subject.

A carrier may be suitable for parenteral administration, which includes intravenous, intraperitoneal or intramuscular administration, Alternatively, the carrier may be suitable for sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated.

Pharmaceutical compositions are typically sterile and stable under the conditions of manufacture and storage. Pharmaceutical compositions may be formulated as a solution, microemulsion, liposome, or other ordered structure suitable to accommodate high drug concentration. The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. A nucleic acid, snRNA or vector of the invention may be administered in a time or controlled release formulation, for example in a composition which includes a slow release polymer or other carriers that will protect the compound against rapid release, including implants and microencapsulated delivery systems. Biodegradable, biocompatible polymers may for example be used, such as ethylene vinyl acetate, polyanhydrides, polyglycolic acid, collagen, polyorthoesters, polylactic acid and polylactic, polyglycolic copolymers (PLG).

The parvoviral, for example AAV, vector of the invention may be of use in transferring genetic material to a cell. Such transfer may take place in vitro, ex vivo or in vivo.

Accordingly, the invention comprises a method for delivering a nucleotide sequence to a cell, which method comprises contacting a composition or a pharmaceutical composition as described herein under conditions such the nucleic acid, snRNA or vector of the invention enters the cell.

The invention also provides a method for administering a nucleotide sequence to a subject, which method comprises administering to the said subject a composition or a pharmaceutical composition as described herein. In particular, the present invention provides a method of administering a nucleic acid of the invention to a subject, comprising administering to the subject a parvoviral gene therapy vector according to the invention together with a pharmaceutically acceptable carrier. Preferably, the parvoviral gene therapy vector is administered in a therapeutically-effective amount to a subject in need thereof.

The invention also provides a nucleic acid, a modified snRNA or a vector of the invention for use in the treatment of the human or animal body by therapy. In particular, a nucleic acid, a modified snRNA or a vector of the invention is provided for use in the treatment of a muscular dystrophy, in particular Duchenne Muscular Dystrophy. A nucleic acid, a modified snRNA or a vector of the invention is provided for use in ameliorating one or more symptoms of a muscular dystrophy, in particular Duchenne Muscular Dystrophy, for example by increasing the formation of dystrophin fibres. Thus, the invention provides a nucleic acid, a modified snRNA or a vector of the invention for use in increasing the production or formation of dystrophin, for example in a cell or in the human or animal body.

That is to say, the invention relates to a method for stimulating or increasing the synthesis, formation or production of dystrophin. Such a method may be carried out ex vivo or in vitro, for example in cells, or in vivo. The amount of dystrophin may be increased in a cell or in a human or animal subject so that the cell or human or animal subject comprises at least about 10%, at least about 20%, at least about 30%, at least about 40%, at least about 50%, at least about 100% of the amount of dystrophin that would be present in a normal, i.e. wild-type, cell or human or animal subject. The amount of dystrophin may be determined at a local or systemic level, i.e. within one, several or all muscles in a human or animal subject. The amount of dystrophin may be expressed as the amount of dystrophin protein or dystrophin fibres.

The invention further provides a method of inducing skipping of an exon in a subject, which method comprises the step of administering an effective amount of a nucleic acid, a modified snRNA or a vector of the invention to the subject.

The invention further provides a method of treatment of a muscular dystrophy, in particular Duchenne Muscular Dystrophy, which method comprises the step of administering an effective amount of a nucleic acid, a modified snRNA or a vector of the invention to a subject in need thereof.

Accordingly, the invention further provides use of a nucleic acid, snRNA or vector as described herein in the manufacture of a medicament for use in the administration of nucleotide to a subject or in inducing skipping of an exon in a subject. Further, the invention provides a nucleic acid, snRNA or vector as described herein in the manufacture of a medicament for use in the treatment of a muscular dystrophy, in particular for use in the treatment of Duchenne Muscular Dystrophy.

Typically, a nucleic acid, snRNA or vector of the invention may be administered to a subject by gene therapy, in particular by use of a parvoviral gene therapy vector such as AAV. General methods for gene therapy are known in the art. The vector, composition or pharmaceutical composition may be delivered to a cell in vitro or ex vivo or to a subject in vivo by any suitable method known in the art. Alternatively, the vector may be delivered to a cell ex vivo, and the cell administered to a subject, as known in the art. In general, the present invention can be employed to deliver any nucleic acid of the invention to a cell in vitro, ex vivo, or in vivo.

The present invention further provides a method of delivering a nucleic acid to a cell. Typically, for in vitro methods, the virus may be introduced into the cell by standard viral transduction methods, as are known in the art.

Preferably, the virus particles are added to the cells at the appropriate multiplicity of infection according to standard transduction methods appropriate for the particular target cells. Titers of virus to administer can vary, depending upon the target cell type and the particular virus vector, and may be determined by those of skill in the art without undue experimentation.

Cells may be removed from a subject, the parvovirus vector is introduced therein, and the cells are then replaced back into the subject. Methods of removing cells from subject for treatment ex vivo, followed by introduction back into the subject are known in the art. Alternatively, an AAV vector may be introduced into cells from another subject, into cultured cells, or into cells from any other suitable source, and the cells are administered to a subject in need thereof.

A further aspect of the invention is a method of treating subjects in vivo with a nucleic acid, snRNA or vector of the invention. Administration of a nucleic acid, snRNA or vector of the present invention to a human subject or an animal in need thereof can be by any means known in the art for administering virus vectors.

A nucleic acid, snRNA or vector of the invention will typically be included in a pharmaceutical composition as set out above. Such compositions include the nucleic acid, snRNA or vector in an effective amount, sufficient to provide a desired therapeutic or prophylactic effect, and a pharmaceutically acceptable carrier or excipient. An “effective amount” includes a therapeutically effective amount or a prophylactically effective amount.

A “therapeutically effective amount” refers to an amount effective, at dosages and for periods of time necessary, to achieve the desired therapeutic result, such as skipping of an exon (so as to lead to protein production to level sufficient to ameliorate the symptoms of the disease associated with a lack of that protein) and/or in increase dystrophin formation or production.

A therapeutically effective amount of a nucleic acid, snRNA or vector of the invention may vary according to factors such as the disease state, age, sex, and weight of the individual, and the ability of the nucleic acid, snRNA or vector to elicit a desired response in the individual. Dosage regimens may be adjusted to provide the optimum therapeutic response. A therapeutically effective amount is also typically one in which any toxic or detrimental effects of the nucleic acid, snRNA or vector are outweighed by the therapeutically beneficial effects.

Viral gene therapy vectors may be administered to a cell or host in a biologically-effective amount. A “biologically-effective” amount of the virus vector is an amount that is sufficient to result in infection (or transduction) and expression of the heterologous nucleic acid sequence in the cell. If the virus is administered to a cell in vivo (e.g., the virus is administered to a subject), a “biologically-effective” amount of the virus vector is an amount that is sufficient to result in transduction and expression of a nucleic acid according to the invention in a target cell.

For a nucleic acid, snRNA or vector of the invention, such as a gene therapy vector, the dosage to be administered may depend to a large extent on the condition and size of the subject being treated as well as the therapeutic formulation, frequency of treatment and the route of administration. Regimens for continuing therapy, including dose, formulation, and frequency may be guided by the initial response and clinical judgment. The parenteral route of injection into the interstitial space of tissue may be preferred, although other parenteral routes, such as inhalation of an aerosol formulation, may be required in specific administration. In some protocols, a formulation comprising the gene and gene delivery system in an aqueous carrier is injected into tissue in appropriate amounts.

Exemplary modes of administration include oral, rectal, transmucosal, topical, transdermal, inhalation, parenteral (e.g., intravenous, subcutaneous, intradermal, intramuscular, and intraarticular) administration, and the like, as well as direct tissue or organ injection, alternatively, intrathecal, direct intramuscular, intraventricular, intravenous, intraperitoneal, intranasal, or intraocular injections. Injectables can be prepared in conventional forms, either as liquid solutions or suspensions, solid forms suitable for solution or suspension in liquid prior to injection, or as emulsions. Alternatively, one may administer the virus in a local rather than systemic manner, for example, in a depot or sustained-release formulation.

The tissue/cell type to be administered a nucleic acid, snRNA or vector of the invention may be of any type, including but not limited to neural cells (including cells of the peripheral and central nervous systems, in particular, brain cells), lung cells, retinal cells, epithelial cells (e.g., vascular, gut and respiratory epithelial cells), muscle cells, such as skeletal muscle or heart muscle, dendritic cells, pancreatic cells (including islet cells), hepatic cells, kidney cells, myocardial cells, bone cells (e.g., bone marrow stem cells), hematopoietic stem cells, spleen cells, keratinocytes, fibroblasts, endothelial cells, prostate cells, adipose deposits, germ cells, and the like. Alternatively, the cell may be any progenitor cell. As a further alternative, the cell can be a stem cell (e.g., neural stem cell, liver stem cell). The tissue target may be specific or it may be a combination of several tissues, for example the muscle and liver tissues.

In the case of a gene therapy vector, the effective dose range for small animals (mice), following intramuscular injection, may be between about 1×10¹¹ and about 1×10¹² genome copy (gc)/kg, and for larger animals (cats) and possibly human subjects, between about 1×10¹⁰ and about 1×10¹³ gc/kg. Dosages of the parvovirus gene therapy vector of the invention will depend upon the mode of administration, the disease or condition to be treated, the individual subject's condition, the particular virus vector, and the gene to be delivered, and can be determined in a routine manner. Typically, an amount of about 10³ to about 10¹⁶ virus particles per dose may be suitable.

The amount of active compound in the compositions of the invention may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation.

It may be advantageous to formulate parenteral compositions in dosage unit form for ease of administration and uniformity of dosage. “Dosage unit form” as used herein refers to physically discrete units suited as unitary dosages for subjects to be treated; each unit containing a predetermined quantity of active compound calculated to produce the desired therapeutic effect in association with the required pharmaceutical carrier. The specification for the dosage unit forms of the invention may be dictated by the unique characteristics of the active compound and the particular therapeutic effect to be achieved, and by the limitations inherent in the art of compounding such an active compound for the treatment of a condition in individuals.

Many methods for the preparation of such formulations are patented or generally known to those skilled in the art.

All publications, patent applications, patents, and other references mentioned herein are incorporated by reference in their entirety.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

The following Examples illustrate the invention:

### EXAMPLES

### Example 1

### Materials and Methods

### Antisense Clones Construction

U1#51 5′3′: this first clone was PCR-derived from the previously described U1-5′ [De Angelis et al. (2002) Proc Natl Acad Sci USA 99: 9456-9461] and double antisense cassette was inserted in the U1 cassette already used for U1#23. Clones U1#51 were obtained by inverse PCR on the construct pRRL-5′3′.

### U1#51 ESE A:

U1#51AON1R
(5′AGAAATGCCATCTTCCTTGAATGAGATCTTGGGCCTCTGC-3′);
U1F2
(5′-GGCAGGGGAGATACCATGATC- 3′).

### U1#51ESEA*:

U1#51ESEA*F
(5′-AGATGGCATTTCTAGGGCAGGGGAGATACCATGATC-3′);
U1#51ESEA*R
(5′-TCCTTGATGTTGGAGATGAGATCTTGGGCCTCTGC-3′)

### U1#51ESEA+B:

U1#51AON1F
(5′ TCAAGGAAGATGGCATTTCTGGCAGGGGAGATACCATGATC-3′)
U1#51AON2R
(5′-ATCAAGTTATAAAATCACAGAGGATGAGATCTTGGGCCTCTGC-
3′).

U1#513′ESEA:

U1#51-3′F
(5′-
GTCTGAGTAGGAGCTAAAATATTTTGGGGGCAGGGGAGATACCATGATC-
3′);
U1#51 AON1R
(5′-AGAAATGCCATCTTCCTTGAATGAGATCTTGGGCCTCTGC-3′)

### U1#515′ESEB:

U1#51ESEBF
(5′-CCTCTGTGATTTTATAACTTGATGGCAGGGGAGATACCATGATC-
3′);
U1#515R
(5′-ATCAAGCAGAAGGTATGAGAAAAAATGAGATCTTGGGCCTCTGC-
3′)

### U1#515′3′Esx:

U1#515′3′sxF
(5′-
TCAAGGAAGATGGGTCTGAGTAGGAGCTGGCAGGGGAGATACCATGATC-
3′);
U1#515′3′sxR
(5′-
TGTTGGAGCATCAAGCAGAAGGTATGAGATGAGATCTTGGGCCTCTGC-
3′)

The PCR fragments were digested with NheI and inserted into the NheI-digested pRRLSIN.cPPT.PGK/GFP.WPRE lentiviral backbone [Bonci et al. (2003) Gene Ther 10: 630-6]. The clone pCCL-MyoD/5′3′ esx was derived from pRRL-5′3′ESX with a digestion reaction using endonucleases SalI and ScaI, and cloning the resulting excised insert into the SalI-ScaI digested pCCL-MyoD (provided by Maurizia Caruso).

### Cell Cultures

Cultures of primary myoblasts were first pre-plated in order to separate fibroblasts from the primary line, seeded in Human Skeletal Muscle Growth Medium (PromoCell, Heidelberg, Germany) and grown in a humidified incubator, at 5% CO₂ and 37° C.

Cultures of primary fibroblasts were established through out-growth from both human DMD and healthy skin biopsies in RPMI with 15% Fetal Calf Serum, 1% penicillin-streptomicine and 1% glutamine (GIBCO/BRL Life Technologies, Grand Island, N.Y., USA). Fibroblasts were then maintained as described above.

### Virus Preparation and Cell Transduction

293T cells were plated on 25-cm diameter plates (3 plates per virus) and co-transfected with lentiviral constructs and packaging plasmids Plp1, Plp2 and PlpV/SVG (Invitrogen, Carlsbad, Calif., USA) according to the four-plasmid transient transfection method previously described [Bonci et al. (2003) Gene Ther 10: 630-6].

Supernatant was collected for the following two days after the transfection and then centrifugated in a Beckmann Ultracentrifuge at 20,000 G for two hours. Pellets were resuspended in HBSS buffer (Invitrogen, Carlsbad, Calif., USA).

Transduction efficiency was tested infecting HeLa cells: cells were then collected for RNA and protein extraction, in order to test antisense and GFP expression respectively. The day before transduction, myoblasts or fibroblasts were seeded in Growth Medium, on 60-mm diameter plates (at least two for each different virus), at a density of 5×10⁵ cells per plate.

The next two days they were infected twice with lentiviruses and polybrene (4 mg/ml).

The second day from the last infection, cells were induced to differentiate with Human Skeletal Muscle Differentiation Medium (PromoCell, Heidelberg, Germany). After 7 days (for myoblasts) or 9 and 13 days (for fibroblasts) of differentiation, cells were washed twice with complete PBS buffer (PromoCell, Heidelberg, Germany) and collected with the help of a gum scraper with 300 microliters of protein buffer (100 mM Tris-HCl pH 7.4, 1 mM EDTA, 2% SDS, 1× Complete EDTA-free Protease Inhibitor Cocktail (Roche)) for protein extraction, or with 1 ml of TRIZOL Reagent (Invitrogen, Carlsbad, Calif., USA) for RNA extraction.

### Proteins Extraction and Analysis

Proteins were extracted from cell plates with 100 mM Tris-HCl (pH 7.4), 1 mM EDTA, 2% sodium dodecyl sulfate (SDS), and a protease inhibitor cocktail (Roche Applied Science, Mannheim, Germany). Samples were placed on a rotary shaker at 4° C. for 30 minutes, centrifugated at 13,200 rpm for 20 minutes at 4° C. and supernatant containing protein extracts was collected. Concentration was assessed with the BCA assay (PIERCE, Thermo Fisher Scientific, Rockford, Ill., USA), according to the manufacturer's instructions.

### Western Blot

50 micrograms of protein extracts (for dystrophin and tubulin) were loaded onto a NuPAGE Tris-Acetate Minigel 3-8% 1 mm (Invitrogen, Carlsbad, Calif., USA); 15 micrograms (for GFP and MyoD) were run on a NuPAGE Bis-Tris minigel 10% 1 mm (Invitrogen, Carlsbad, Calif., USA).

Running and blotting were performed in an XCell SureLock™ Minicell (Invitrogen, Carlsbad, Calif., USA) according to the manufacturer's instructions and proteins were transferred to a nitrocellulose transfer membranes (Protran; Schleicher & Schuell BioScience, Keene, N.H., USA).

Membranes were blocked with 10% non-fat dry milk (Difco™ Skim Milk, Becton & Dickinson, Le Pont de Claix, France), incubated for 1 h with primary antibody (for dystrophin: NCL-DYS1 (Novocastra, Newcastle upon Tyne, UK) diluted 1:40 in 3% milk; for tubulin: anti-tubulin AA12 (Santa Cruz Biotechnology Inc., Santa Cruz, Calif., USA) diluted 1:200 in TBST; for GFP: anti-GFP ab290 (AbCAM, Cambridge, UK) diluted 1:2500 in TBST; for MyoD: anti-MyoD 5.8 A (Santa Cruz Biotechnology Inc., Santa Cruz, Calif., USA), diluted 1:500 in 2% milk), washed in (TBST) Tris-buffered saline with Tween 20 (Sigma Aldrich®, St. Louis, Mo., USA), and incubated with goat anti-mouse IgG (H+L) horseradish peroxidase (HRP)-conjugated secondary antibody (diluted 1:5000 in 3% milk) for dystrophin, tubulin and MyoD, or ImmunoPure®Goat Anti-Rabbit IgG Peroxidase Conjugated (PIERCE, Thermo Fisher Scientific, Rockford, Ill., USA) for GFP for 1 hr. Protein detection was carried out with SuperSignal chemiluminescent substrate (PIERCE, Thermo Fisher Scientific, Rockford, Ill., USA).

### RNA Preparation and Analysis

Cells were harvested with 1 ml of Trizol (Invitrogen, Carlsbad, Calif., USA) and RNA was extracted according to the manufacturer's instructions; concentration was assessed with Nanodrop ND-1000 Spectrophotometer (CELBIO, Italy).

### RT-PCR

Dystrophin mRNA was analyzed by RT-PCR on 200 ng of total RNA with oligos E46F (5′-GCTAGAAGAACAAAAGAATAT-3′) for deletion 48-50 or oligo E43F (5′-CTACAACAAAGCTCAGGTCG-3′) for deletion 45-50 and E54R (5′-CTTTTATGAATGCTTCTCCAAG-3′), for 40 cycles with the Access RT-PCR system (Promega, Madison, Wis., USA). Four microliters of the RT-PCR products were then used as template for the nested reaction performed with oligo E47F (5′-TTACTGGTGGAAGAGTTGCC-3′) and E52Ro (5′-TTCGATCCGTAATGATTGTTCTAGCC-3′) for 30 cycles.

10 microliters of the reactions were run on a 2% agarose-ethidium bromide gel and signals were revealed on a UV transilluminator.

### Northern Blot

Northern analysis was performed as already described [Rivera et al. (2005) Blood 105: 1424-30]. Briefly, 10 micrograms of total RNA were loaded onto a 6% polyacrilammide gel, run at 17 mA and transferred into a Hybond™-N⁺ nitrocellulose membrane (Amersham, GE Healthcare Life sciences, Buckinghamshire, UK) for 16 h at 10 volts and 4° C. Membranes were hybridized with probe α-U1 (5′-CAGGGGAAAGCGCGAACGCAGTCCCCCA-3′) and revealed using a Typhoon TRIO Variable Mode Imager system (Amersham, GE Healthcare Life sciences, Buckinghamshire, UK), with ImageQuant TL.I program.

pLuc-ex51 Construction

The insert ex51 was obtained by PCR from genomic DNA using the oligos: 5′-AAGGAAAAAAGCGGCCGCATGAGAATGAGCAAAATCGT-3′ and 5′-CCTTAATTAAGAGACAACTATTCTTGTAAG-3′.

The underlined are NotI and Pad restriction sites.

Ex51 is made of the whole exon 51 flanked by 268 bp of intron 50 and 263 bp of intron 51. The PCR fragments were digested with NotI and Pad enzymes and inserted into the NotI and PacI-digested pcDNA3.1-Luc vector [Bian et al. (2009) Hum Mol Genet. 18: 1229-37].

### C₂7 Transfections

C2.7 myoblasts [Pinset et al. (1988) Differentiation 38: 28-34] are a mouse myogenic cell line derived from the C2 cells, isolated from mouse activated satellite cells [Yaffe and Saxel (1977) Nature 270: 725-7]. C₂7 cells were plated on 35-mm diameter plates; they were co-transfected with 2 μg of pLuc-ex51, 2 μg of the lentiviral vector carrying the antisense expression cassette and 50 ng of a Renilla Luciferase expressing construct, used as a transfection efficiency control. The transfection was performed according to the Lipofectamine 2000 protocol (Invitrogen, Carlsbad, Calif., USA). Cells were grown in DMEM 10% FBS for 36 hours.

### Luciferase Assay

C₂7 cells were collected using 250 μL of Passive Lysis Buffer and the assay was performed according to Dual-Luciferase Reporter Assay System protocol (Promega, Madison, Wis., USA).

### Design and Expression Analysis of Antisense Molecules Against Exon 51 of the DMD Gene

The U1 snRNA was utilized as carrier to express 7 different antisense molecules for exon 51 skipping. Nucleotides from position 3 to 10 at the 5′ end of U1 snRNA, required for the recognition of the 5′ splice site, were substituted with antisense sequences complementary to different portions of exon 51 and its splice sites (FIG. 1a). Since we previously observed that both splice sites have to be targeted in order to induce efficient exon skipping [De Angelis et al. (2002) Proc Natl Acad Sci USA 99: 9456-9461], the first constructs produced contained antisense sequences against both splice junctions (5′3′ constructs—FIG. 1b). Moreover, since Exonic Splicing Enhancers (ESEs) have been shown to represent effective target substrates for efficient exon skipping [Aartsma-Rus et al. (2005) Oligonucleotides 15: 284-297; Aartsma et al. (2006) Ann N Y Acad Sci 1082:74-6] we also produced chimeric constructs containing antisense sequences against putative ESE elements [Cartegni et al. (2003) Nucleic Acids Res 31: 3568-3571], alone or in combination with splice junctions (FIG. 1b).

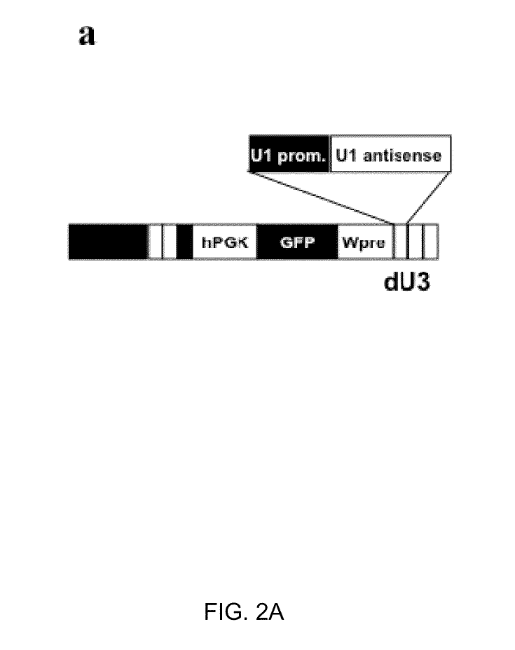

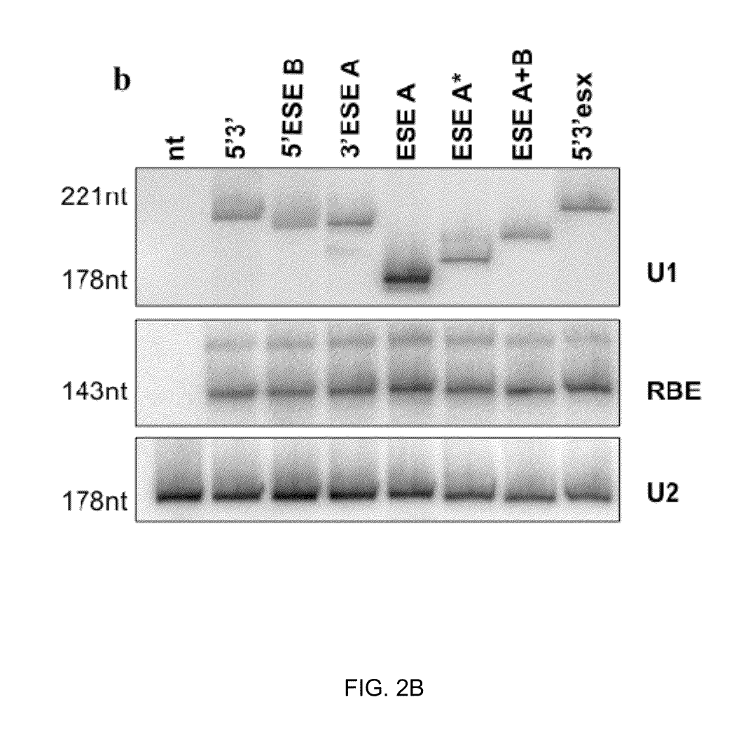

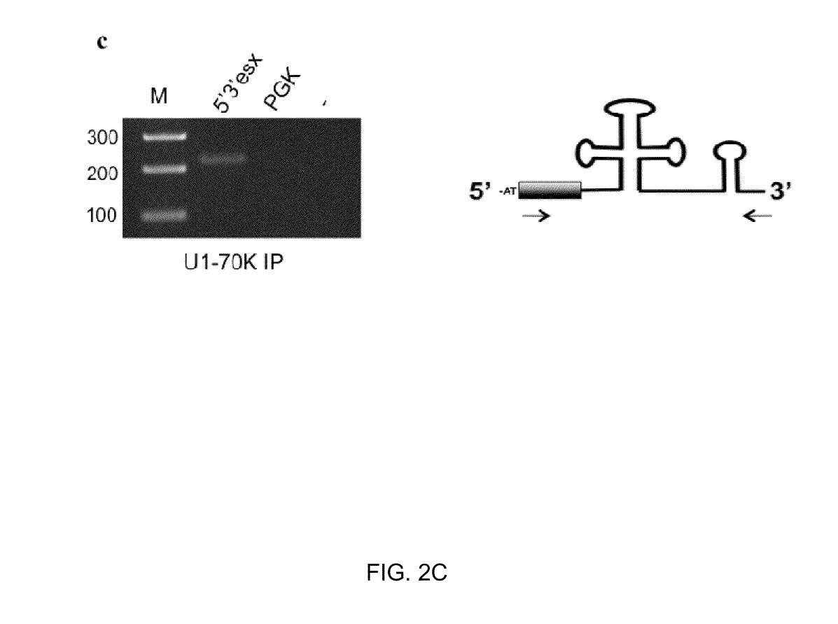

The strong polymerase II-dependent U1 snRNA gene promoter and termination sequences were used to derive antisense expression cassettes, which were cloned in the dU3 portion of the 3′ Long Terminal Repeat (LTR) region of the pRRLSIN.cPPT.PGK/GFP.WPRE lentiviral vector [Bonci et al. (2003) Gene Ther 10: 630-6] (FIG. 2a). Since the different constructs considerably extend the length of the U1 snRNA (in some cases up to 58 nucleotides), we first checked their expression and stability in HeLa transfection experiments. The relative expression activity was tested by co-transfection with the U16RBE plasmid [Buonomo et al. (1999) RNA 5: 993-1002] and normalized for the endogenous U2 snRNA. Northern blot analysis indicated that all the chimeric molecules accumulated at fairly similar levels (FIG. 2b). Moreover, immunoprecipitations of nuclear extracts with U1-70K antibodies followed by RT-PCR (FIG. 2c), indicated that the U1-chimeric snRNAs are still able to form snRNPs [Lerner and Steitz (1979) Proc Natl Acad Sci USA 76: 5495-9].

### Results

### Study of Exon Skipping Activity in Human DMD Myoblasts

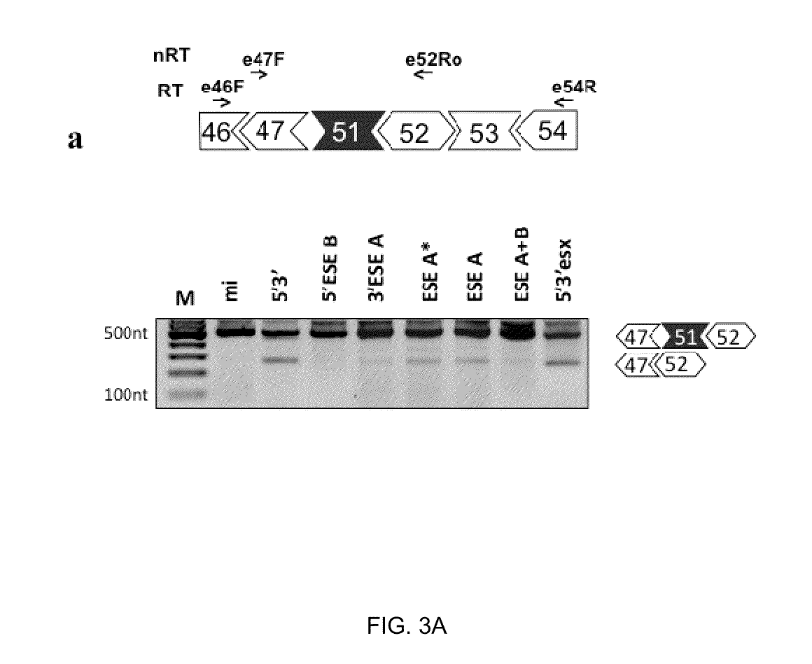

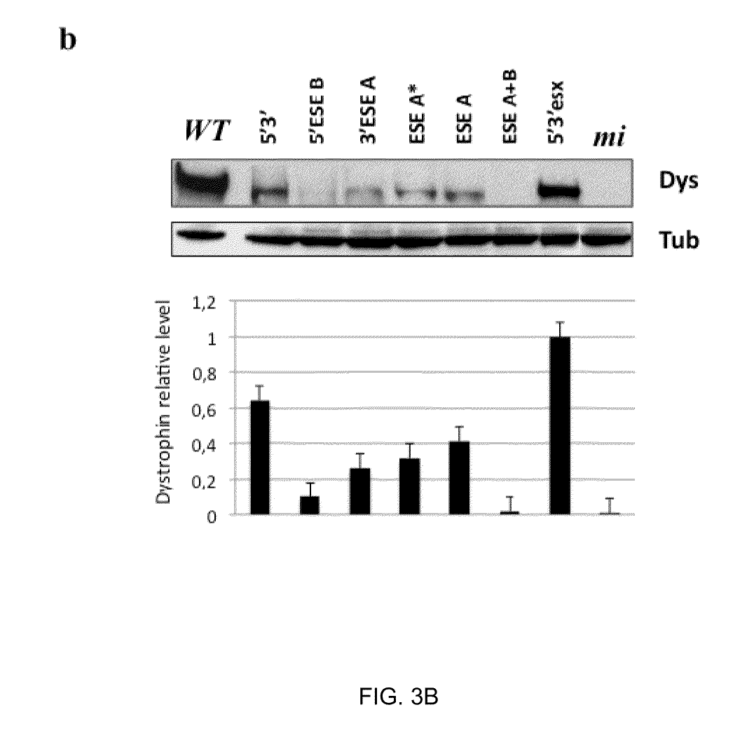

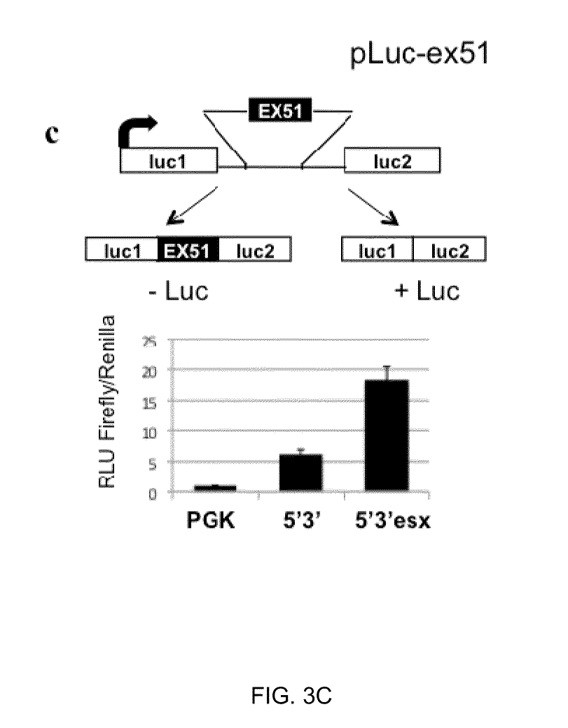

Lentiviral particles for each of the constructs were produced in 293T cells and used to infect DMD myoblasts (Δ48-50) carrying the deletion of exons 48-50 (provided by the Telethon Neuromuscular Biobank). In this case, skipping of exon 51 allows rescue of the correct reading frame and the production of a dystrophin protein 210 amino acids shorter than the wild type. Δ48-50 cells were infected with comparable amounts of the different recombinant lentiviruses. After infection, they were induced to differentiate and samples for RNA and protein analysis were collected after 7 days. Exon 51 skipping was evaluated by nested RT-PCR analysis performed on 200 ng of total RNA (FIG. 3a), while Western blot was performed on 50 μg of total proteins (FIG. 3b). Dystrophin levels, measured in three independent experiments, were normalized against the tubulin signal; the relative values are reported in the histogram of FIG. 3b. With the exception of 5′ESE B and ESE A+B constructs, all the samples revealed skipping activity and, among these, 5′3′ and 5′3′ esx were the most effective. Notably, the skipping activity paralleled very well the efficiency of dystrophin rescue (FIG. 3b), with constructs 5′3′ and 5′3′ esx displaying the highest activity.

Comparison between the 5′3′ and 5′3′ esx constructs was further tested using a luciferase-based splicing reporter assay [Bian et al. (2009) Hum Mol Genet. 18: 1229-37] in which luciferase is produced only when skipping of the inserted exon 51 is occurring (FIG. 3c, upper panel—construct pLuc-ex51). 5′3′ and 5′3′ esx expressing plasmids were co-transfected with the pLuc-ex51 and with a Renilla reporter. Luciferase activity assays (FIG. 3c, lower panel) indicated that 5′3′ esx is inducing a higher level of exon skipping with respect to 5′3′, confirming the data obtained in DMD cells. These results indicated that the ESE target region present in the 5′3′ esx construct, plays an important role in exon 51 splicing and that it is able to improve skipping efficiency when combined to antisense sequences against splice junctions.

Exon Skipping in DMD Fibroblasts Trans-Differentiated into Myoblasts

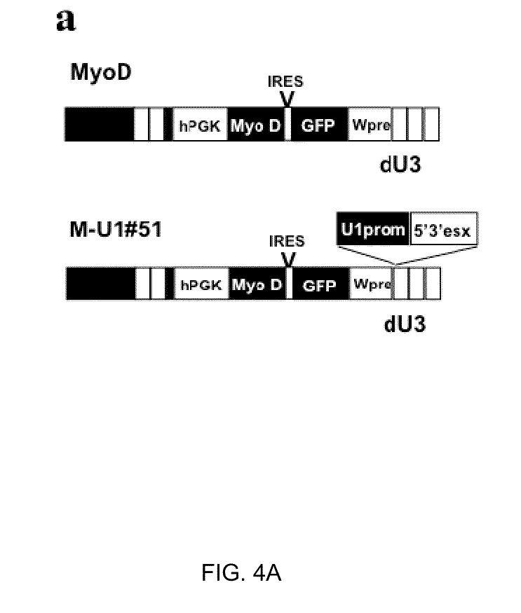

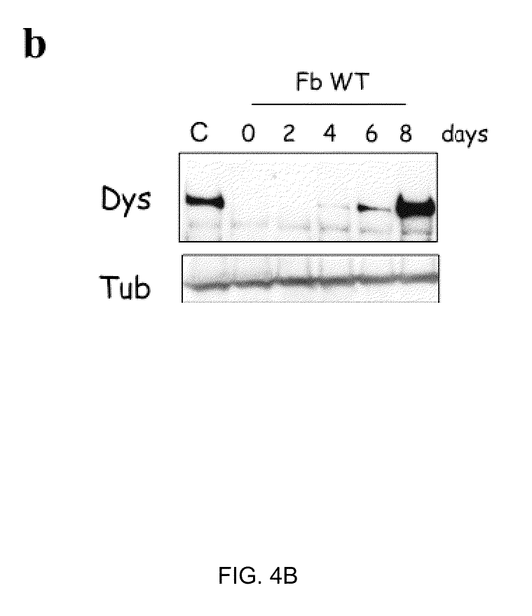

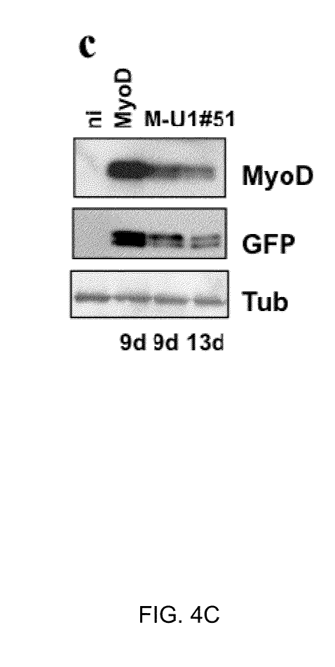

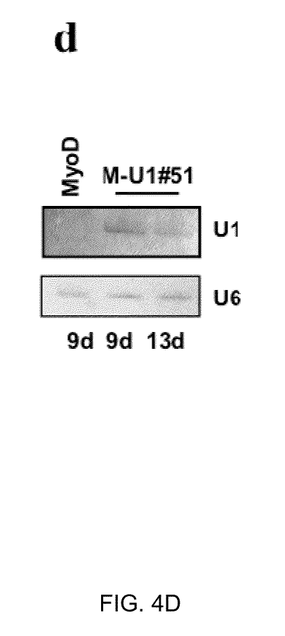

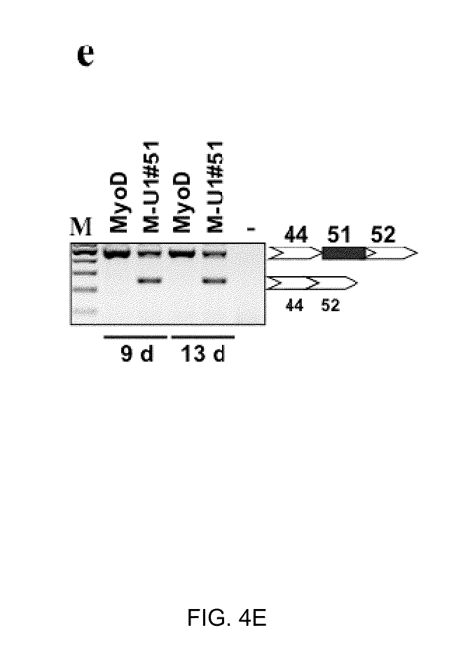

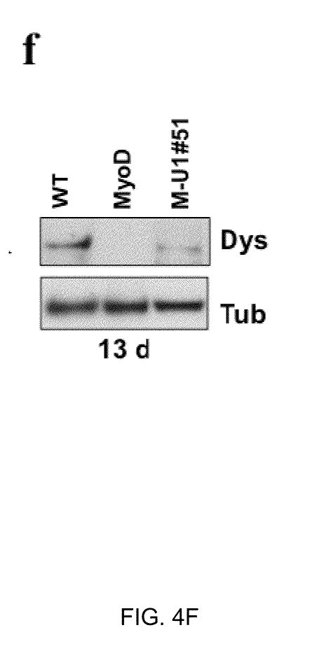

In order to have a simplified screening procedure for testing the activity of antisense constructs on patients' samples, we set up a protocol for fibroblasts trans-differentiation into myoblasts. Infection of WT fibroblasts with the MyoD lentivirus (FIG. 4a) produced efficient conversion into myoblasts and myotubes as shown by dystrophin synthesis (FIG. 4b) and morphological analysis (not shown). Dystrophin starts to be produced at 4 days after shifting to differentiation medium and its accumulation increases with the progression of the myogenic program (8 days). We then applied the trans-differentiation protocol to DMD fibroblasts in order to test the skipping activity of the 5′3′ esx construct. A skin biopsy with a different genetic background, (deletion of exons 45-50-Δ45-50), also treatable with exon 51 skipping, was infected with the M-U1#51 lentivirus carrying the MyoD and 5′3′ esx expression cassettes (FIG. 4a). Muscle differentiation was induced two days after infection and at day 9 and 13, samples were collected for RNA and protein analysis. FIG. 4c shows the expression of MyoD and GFP which are encoded on the same vector. FIG. 4d shows that the expression of the antisense RNA persisted at 9 and 13 days and it corresponded to a very high efficiency of skipping (almost 50%, FIG. 4e) and rescue of dystrophin synthesis (FIG. 4f).

### Discussion

Exon skipping is one of the most promising gene therapy approaches for the treatment of DMD. Two phase I clinical trials have been concluded using different kinds of modified-antisense oligonucleotides delivered to the patients by local intramuscular injections. In both studies, the analysis performed 3-4 weeks after treatment revealed a high percentage of dystrophin positive fibres and proved to be safe and well tolerated.

A parallel strategy to the use of synthetic oligonucleotides is represented by a gene therapy approach in which the antisense molecule is continuously expressed as part of a cellular RNA. Several groups have indeed proven the feasibility of this approach through the use of expression cassettes for snRNA-based antisense molecules delivered to mdx mice muscle cells by AAV vectors. Further studies in the mdx model have also shown that a single systemic injection into young (6-week old) animals of AAV1-U1 antisense RNA constructs was effective in maintaining the physiological and molecular benefits for the entire life span of the animals. These results encouraged the idea that one single treatment could be effective in providing a long term benefit and provided the basis for designing protocols with potential applications of AAV-mediated gene therapy in human.

The objective of present work was the selection of the most effective antisense-RNA for the skipping of exon 51 of the human DMD gene.

Seven different U1 snRNA-based constructs combining antisense sequences directed against splice junctions and exonic splicing enhancers were designed, cloned and tested for in vivo stability and exon 51 skipping activity. All of them were shown to stably accumulate in the cell despite the fact that the U1 snRNA size was sometime considerably modified by the addition of the antisense sequences. Notably, these molecules were still able to bind the U1-70K protein which represents a hallmark for the assembly of stable U1 snRNP particles, thus explaining the reason for their stable accumulation in the cell.

The exon 51 skipping behaviour of each antisense molecule was tested by infecting myoblasts derived from patient biopsies with the deletion of exons 48-50. This allowed us to select the two best performing molecules (5′3′ and 5′3′ esx), differing only for an additional antisense element against a putative ESE in the 5′3′ esx construct. Subsequently, their activity was further compared using a luciferase-based splicing-reporter assay that confirmed the higher activity of the 5′3′ esx construct. These data indicated that the ESE target region present in the 5′3′ esx construct, plays an important role in exon 51 splicing and that, in the U1 snRNA context, is able to improve skipping efficiency when associated to antisense sequences against splice junctions. These results, together with the fact that the U1 constructs containing only anti-ESE antisense have very poor activities, allowed us to conclude that U1 snRNA works efficiently primarily through recognition of splice junctions. This appears a relevant difference to synthetic oligos which act independently of any carrier RNA.

The activity of the winner construct was finally tested on a different genetic background (deletion 45-50) utilizing trans-differentiated patients' fibroblasts. The possibility of testing exon skipping activity in fibroblasts derived from skin biopsies is very important, considering that muscle biopsies are invasive surgical interventions and DMD patients are basically children, already weakened by the course of their pathology.

In conclusion, the results of these studies indicated that the 5′3′ esx construct is very active in inducing skipping of exon 51 in two different DMD mutant backgrounds and in two different cellular systems.

1. A nucleic acid encoding a small nuclear RNA (snRNA), which snRNA is modified so that it comprises sequence capable of hybridizing with the 5′ and 3′ splice site junctions and an exonic splicing enhancer sequence of an exon of a pre-mRNA, so that the exon sequence is skipped during the splicing process that converts the pre-mRNA into a mature mRNA.

2. A nucleic acid according to claim 1, wherein the exon is an exon of the dystrophin pre-mRNA.

3. A nucleic acid according to claim 2, wherein the exon is exon 51 of the dystrophin pre-mRNA.

4. A nucleic acid according to claim 1, wherein the sequence capable of hybridizing with the 5′ and 3′ splice site junctions and to an exonic splicing enhancer of an exon of the dystrophin pre-mRNA is comprised with the 5′ region of the snRNA.

5. A nucleic acid according to claim 1, wherein the modified snRNA is a modified U1 snRNA.

6. A nucleic acid according to claim 5, wherein the modified U1 snRNA retains the ability to interact with the U1 associated 70K protein.

7. A nucleic acid according to claim 3, wherein the sequence capable of hybridizing with the 5′ splice junction of exon 51 of the dystrophin pre-mRNA comprises sequence capable of hybridizing with SEQ ID NO: 1.

8. A nucleic acid according to claim 3, wherein the sequence capable of hybridizing with the 3′ splice junction of exon 51 of the dystrophin pre-mRNA comprises sequence capable of hybridizing with SEQ ID NO: 2.

9. A nucleic acid according to claim 3, wherein the sequence capable of hybridizing with the exonic splicing enhancer of exon 51 of the dystrophin pre-mRNA comprises sequence capable of hybridizing with SEQ ID NO: 3.

10. A nucleic acid according to claim 3, wherein the sequence capable of hybridizing with the 5′ and 3′ splice junctions and an exonic splicing enhancer of exon 51 of the dystrophin pre-mRNA comprises the sequence of SEQ ID NO: 4.

11. A nucleic acid according to claim 1, which comprises the sequence of SEQ ID NO: 5.

12. A nucleic according to claim 1, wherein skipping of the exon by the modified snRNA leads to the production of a functional protein.

13. A modified snRNA encoded by a nucleic acid according to claim 1.

14. A vector which incorporates a nucleic acid according to claim 1.

15. A vector according to claim 14, which is a gene delivery vector.

16. A vector according to claim 15, which is a viral gene delivery vector or a non-viral gene delivery vector.

17. A vector according to claim 16, wherein the viral gene delivery vector is based on a parvovirus, an adenovirus, a retrovirus, a lentivirus or a herpes simplex virus.

18. A vector according to claim 17, wherein the viral gene delivery vector based on a parvovirus is an adenovirus-associated virus.

19. A mammalian or insect cell comprising a nucleic acid according to claim 1.

20. A method for the preparation of a parvoviral gene delivery vector which method comprising the steps of:
(a) providing an insect cell comprising one or more nucleic acid constructs comprising:
(i) a nucleic acid according to claim 1 that is flanked by at least one parvoviral inverted terminal repeat nucleotide sequence;
(ii) a first expression cassette comprising a nucleotide sequence encoding one or more parvoviral Rep proteins which is operably linked to a first promoter that is capable of driving expression of the Rep protein(s) in the insect cell;
(iii) a second expression cassette comprising a nucleotide sequence encoding one or more parvoviral capsid proteins which is operably linked to a second promoter that is capable of driving expression of the capsid protein(s) in the insect cell;
(b) culturing the insect cell defined in (a) under conditions conducive to the expression of the Rep and the capsid proteins; and, optionally,
(c) recovery of the parvoviral gene delivery vector.

21. A pharmaceutical composition comprising a nucleic acid according to claim 1 and a pharmaceutically acceptable carrier or diluent.

22. A nucleic acid according to claim 1, suitably formulated in a pharmaceutical composition.

23. A method of treatment of a muscular dystrophy, which method comprises the step of administering an effective amount of a nucleic acid according to claim 2 to a subject in need thereof.

24. A mammalian or insect cell comprising a modified snRNA according to claim 13.

25. A mammalian or insect cell comprising a vector according to claim 14.

26. A pharmaceutical composition comprising a modified snRNA according to claim 13 and a pharmaceutically acceptable carrier or diluent.

27. A pharmaceutical composition comprising a vector according to claim 14 and a pharmaceutically acceptable carrier or diluent.

28. A modified snRNA according to claim 13 suitably formulated in a pharmaceutical composition.

29. A vector according to claim 14 suitably formulated in a pharmaceutical composition.

30. The method of claim 23, wherein the muscular dystrophy is Duchenne muscular dystrophy.

31. A method of treatment of a muscular dystrophy, which method comprises the step of administering an effective amount of a modified snRNA according to claim 13.

32. A method of treatment of a muscular dystrophy, which method comprises the step of administering an effective amount of a vector according to claim 14.


SEQUENCE LISTING (5 sequences)

SEQ ID NO: 1 - DNA, 20 bp, Homo sapiens
catcaagcagaaggtatgag

SEQ ID NO: 2 - DNA, 16 bp, Homo sapiens
tagctcctactcagac

SEQ ID NO: 3 - DNA, 21 bp, Homo sapiens
ccatcttccttgatgttggag

SEQ ID NO: 4 - DNA, 56 bp, Homo sapiens
ctcataccttctgcttgatgctccaacatcaaggaagatgggtctgagtaggagct

SEQ ID NO: 5 - DNA, 212 bp, Homo sapiens
atctcataccttctgcttgatgctccaacatcaaggaagatgggtctgagtaggagctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctg